(19) United States
(12) Patent Application Publication
(10) Pub. No.: US 2026/0337652 A1
(43) Pub. Date: Oct. 29, 2026

(54) CRISPR-CAS9-BASED LINEARIZATION OF PLASMID DNA TEMPLATES

(71) Applicant: Wacker Chemie AG, Munich (DE)

(72) Inventors: Hagen Richter, Munich (DE); Carsten Bornhoevd, Munich (DE); Philippe Cronet, Munich (DE); Marian Kujau, Munich (DE); Guenter Wich, Munich (DE)

(73) Assignee: Wacker Chemie AG, Munich (DE)

(21) Appl. No.: 19/107,122

(22) PCT Filed: Mar. 29, 2023
(86) PCT No.: PCT/EP2023/058156

§ 371 Date: Feb. 27, 2025

(51) Int. Cl. C12P 19/34

(52) U.S. Cl. CPC C12P 19/34

(57) ABSTRACT

A method for producing a linear double-stranded DNA molecule and uses for the same. Where the linear DNA molecule has blunt ends and the linear DNA molecule has a poly T sequence at the 5′ end of the template strand, using a guide DNA and a Cas nuclease.

The invention relates to a method for producing a linear double-stranded DNA molecule, the linear DNA molecule having blunt ends, and the linear DNA molecule having a poly T sequence at the 5′ end of the template strand, comprising the steps of:
(1) providing a double-stranded circular DNA molecule comprising on the template strand in the 3′ to 5′ direction:
(i) a template sequence operatively linked to an RNA polymerase promoter,
(ii) a poly T sequence,
(iii) a nucleotide sequence described by N₁N₂N₃, where N₁ to N₃ are each independently G, T, A or C or a nucleotide analog thereof, and
(iv) a protospacer adjacent motif (PAM),
(2) providing a guide RNA comprising a region which is complementary to the 5′ end of the poly T sequence (ii) and to the nucleotide sequence (iii) and a region which interacts with a Cas nuclease,
(3) providing a type II Cas nuclease,
(4) contacting the double-stranded circular DNA molecule with the guide RNA and the CAS nuclease.

Ribonucleic acids (RNAs) play a crucial role in many cellular processes. According to the central dogma of biology, they mediate the rendering of DNA into proteins and play the key role in transcription and translation in the form of messenger RNAs (mRNAs) and transfer RNAs (tRNAs). But many other processes, especially at the regulatory level, i.e., the fine-tuning of biochemical reactions inside and outside cells, are also significantly controlled by RNAs. It is therefore not surprising that RNAs are a focus of a multitude of research work for better understanding of these processes. In addition to fundamental work on a general understanding of RNAs, what has been found in particular in recent years is how useful RNAs are for therapeutic applications as well, for example vaccines against SARS-Cov2. For such applications, it is important that suitable methods allowing production of sufficient amounts of mRNA are available. Said methods use naturally occurring molecules, for example RNA polymerases, which produce RNAs. The synthetic production of RNAs by DNA-dependent RNA polymerases is a method routinely used for research applications and industrial applications. Said method requires a DNA template, an RNA polymerase, for example a T7 RNA polymerase, nucleotides such as ATP, CTP, GTP and UTP, and suitable buffer conditions. The polymerase then synthesizes a corresponding complementary RNA according to the DNA template. The RNAs produced can differ in type: mRNAs, self-amplifying RNAs (saRNAs), long noncoding RNAs (lncRNAs), guide RNAs (gRNAs), short interfering RNAs (siRNA), microRNAs (miRNAs), circular RNAs (circRNAs) and others. Especially in the case of relatively short RNAs, chemical synthesis can also be used industrially. However, chemical synthesis is not economical for relatively long RNAs of more than 100 nucleotides, for example mRNAs, and as a consequence, relatively long RNAs are usually produced by enzymatic in vitro transcription.

In large-scale production, plasmid DNA is generally used as DNA template for in vitro transcription, since plasmid DNA, unlike DNA templates produced by PCR, can be produced in the necessary amounts by fermentation. The self-contained, circular nature of plasmids leads to a theoretically infinitely long RNA transcript in RNA synthesis; in practice, it always leads to at least a proportion of unwantedly heterogeneous and extended RNAs. These extensions have an adverse effect on the efficiency of the transcription process, thereby increasing the production costs. Two possible approaches to avoid the unwanted extended RNA transcripts are described in the literature.

One solution is the classic use of type II restriction endonucleases. These enzymes recognize a specific DNA sequence and generally make a specific cut in the DNA double strand within or near said sequence. Such a cut in the plasmid DNA allows so-called in vitro run-off transcription, in which the RNA polymerase “falls off” at the double-strand break and can rebind to a promoter and start a new round of synthesis. Classic type II restriction enzymes are typically used for such a cut in the DNA template. Restriction enzymes of the subgroup type IIP cut DNA at defined positions within the recognition sequences thereof. Examples of type IIP restriction enzymes are EcoRI and XbaI. Alternatively, use can also be made of enzymes of the subgroup type IIS, which cut outside the recognition sequences thereof. The two classes of type II restriction enzyme (type IIP and type IIS) can be used to generate linear DNA fragments from the circular plasmid DNA, and the former can be used for more efficient RNA synthesis. It should be noted here that the DNA template should not contain any further recognition sequences for the particular restriction enzymes used. Thus the template sequence is subject to a constraint and its design is more complex. This can affect individual base pairs, but it can also affect entire codons of the DNA template or domains of the target proteins, for example motifs or structures. In addition, many type II restriction enzymes, especially type IIS enzymes, process the DNA with an overhang, forming so-called sticky ends at the 5′ or 3′ end. A 3′ overhang is particularly unfavorable, since it can lead to longer transcripts than originally planned owing to the promiscuity of the RNA polymerase. In order to prevent these unwanted transcripts, the overhang would need to be degraded following the restriction step. Thus there are distinctly more steps in planning and generating DNA templates, and there is an increase in the corresponding amount of development required.

The second solution to avoid excessively long RNA transcripts is the use of Rho-independent termination signals. In contrast to use of restriction endonucleases, there is no linearization of the DNA template in this case. Rho-independent terminators are usually secondary structures within the synthesized target RNA and prevent excessively long RNA transcripts from forming by termination of synthesis. After synthesis has been terminated, the polymerase, similar to run-off transcription, can rebind a promoter and start a new round of synthesis. A high rate of transcription is maintained, but sequence regions that are undesirable, for example the terminator sequence, are also added. In order to remove these undesirable elements, use can be made of not only RNA-cutting enzymes, for example RNases, but also catalytically active nucleic acids. Examples thereof are DNAzymes, but also so-called ribozymes. Ribozymes are structured RNA molecules that cut RNAs at defined sites in trans (coming from outside) or in cis (coming from the sequence). This generally requires a specific RNA structure and a divalent metal ion, typically Mg²⁺. This results in a nucleophilic attack on the target sequence, as a result of which it is cleaved and, in the case of RNA synthesis, excess elements can be removed. Since T7 RNA polymerase is also dependent on Mg²⁺ ions, the ribozyme activity within the synthesis reaction will have an influence on the efficiency of in vitro transcription owing to competition for Mg²⁺ ions. Thus further optimization of the reaction conditions is required, which may be associated with losses in efficiency. When designing appropriate sequences having a Cis-ribozyme, it must be ensured that a ribozyme followed by at least one terminator sequence is incorporated into the target RNA. Accordingly, the entire process for producing DNA templates for mRNA production becomes more costly and thus less economical.

For the enzymatic production of functional mature mRNAs and saRNAs that are to be active in mammalian cells, it is an absolute requirement that the RNA have a poly A tail. Said poly A tail increases RNA stability and also plays a crucial role during translation in the eukaryotic target cell. In the target cell, the poly A tail is recognized and bound by poly A-binding proteins. The bound proteins cause the interaction of the 3′ end of the mRNA with the proteins of the translation machinery on the 5′ end of the mRNA. This complex interplay increases not only RNA stability, but also translation efficiency. A key role is therefore played by the poly A tail within the mature mRNA. In order to provide mRNA or saRNA that is as natural as possible, the poly A tail should be as long as possible (>100 A nucleotides) and ideally homogeneous (without any other bases; C, G or U). The poly A tail at the 3′ end of the RNA is then referred to as an uncovered poly A tail.

There are currently two customary ways of producing RNA molecules having a uniform poly A tail. Firstly, the synthesis of the target RNA sequence can be followed by the generation of the poly A tail by means of a poly A polymerase. In the case of this enzymatic step, the RNA produced is first purified, before a poly A tail is synthesized by addition of a poly A polymerase and ATP as substrate. The length of time for the reaction determines the average length of the poly A tails. This enzymatic synthesis generally works very well, but also has a few disadvantages. For instance, it is not possible with this technique to generate a clearly defined length of the poly A tail, and what is formed is a product comprising inhomogeneous RNA molecules with a normal distribution of their lengths. Industrial use of the RNA molecules thus produced therefore requires further process steps for enrichment or purification of the RNAs, and this lowers yields and increases production costs.

In contrast to this is a method in which the poly A tail is directly encoded by the DNA template serving as template for production of the RNA. For this purpose, for polyadenylation, it must be ensured when designing the template DNA that the double-stranded DNA contains the necessary poly T motif on the template strand. This method has the advantage of forming, within one reaction step, a much more defined product having a poly A tail of uniform length.

If PCR products are used as template for the synthesis of RNA molecules having homogeneous and uncovered poly A tails, the poly A tail can be directly coded in the primers used for the PCR. To produce a DNA template by PCR, use can be made of a reverse primer containing, in addition to the complementary sequence at the 3′ end, a succession of up to 120 or more adenines. In the PCR, this tail is added to the coding DNA and is therefore available for in vitro transcription. Since PCR reactions can technically usually only be carried out in small volumes of up to a few hundred microliters, large-scale processing to produce RNA would require several hundred PCR reactions. Upscaling of the PCR reaction by means of so-called scale-out, in which multiple reactions are carried out in parallel, is currently not economical, since the work and cost involved on an industrial scale is high.

If plasmid DNA is used as template for the in vitro transcription of RNA, this requires fermentative production of the plasmids, purification of the pDNA and subsequent linearization of the pDNA. After linearization, the linearized pDNA must be repurified to provide the purest possible DNA template for in vitro transcription, thereby increasing 15 the complexity and costs of the method. As already noted, a disadvantage of using type IIP restriction enzymes is that these enzymes do not yield a free uncovered poly A tail, since the recognition sequence of type IIP restriction enzymes almost always contains other nucleotides as well, such as C, G or T. In this regard, a remedy could be provided by the use of type IIS enzymes, since they would be able to cut outside their recognition sequence, thus creating the desired uncovered poly A tail. However, the use of type IIS enzymes usually leads to 3′ and 5′ overhangs, which, as described above, would first need to be degraded.

Besides the use of restriction enzymes, it is also possible to use ribozymes for linearization of template DNA. These too can be designed such that a homogeneous and free poly A tail can be formed. Ribozymes are preferably designed to directly cut at the end of the poly A sequence. The disadvantage of ribozymes is that a further RNA has to be produced in the case of trans ribozymes and a distinctly longer RNA has to be produced in the case of cis ribozymes. This approach increases the complexity of production and accordingly the costs.

CRISPR (Clustered Regularly Interspaced Short Palindromic Repeats) and CRISPR-associated (Cas) nucleases have been identified as components of bacterial immune systems. Since Cas nucleases are able to cut DNA elements precisely at a specific site, CRISPR-Cas systems have been developed as efficient gene editing tools, mainly for applications in the field of genome editing, and have led to a revolution in molecular biology and microbiology in this field (Hille et al., Cell 172, Issue 6, 1239-1259, 2018; Plagens et al. FEMS Microbiology Reviews, 39 (3): 442-63, 2015; Richter et al. Int J Mol Sci. 2013 July; 14 (7): 14518-14531).

If a bacterial cell is infected by a foreign nucleic acid, for example phage DNA, transposons, pDNA or RNA, the bacterium reacts with an immune response in order to eliminate the foreign DNA or RNA. CRISPR-Cas immunity is remarkable in that the immunity is adaptive immunity, which means that this type of immune response is learnt by the bacterial cell. During an infection, a short DNA fragment of the foreign nucleic acid is used for the immune system, as a result of which a more rapid response can be induced later in the event of an identical type of infection. This DNA fragment is incorporated within the genetic locus for the CRISPR-Cas system in the CRISPR array, where it is referred to as a spacer. This spacer sequence later becomes part of the guide RNA (gRNA) or CRISPR RNA (crRNA) and thus contributes to sequence specificity. As part of this bacterial immune defense, the Cas nuclease is directed by the crRNA to a specific target sequence on the foreign nucleic acid. The recognition of the sequence requires, firstly, the complementarity of the crRNA with the foreign target DNA and, secondly, a short signal sequence in the direct vicinity of the target DNA, which is referred to as protospacer adjacent motif (PAM). Depending on the CRISPR-Cas system, the PAM sequence consists of a varying number of specific nucleotides and is the initial recognition sequence for the Cas nuclease on the DNA to be cut. For example, the Cas nuclease Cas9 from Streptococcus pyogenes (SpCas9) recognizes the PAM sequence 5′-NGG-3′. If there is no PAM sequence in the direct vicinity of the target sequence, the foreign DNA is not cut. After it has been recognized by the ribonucleoprotein complex of Cas nuclease and crRNA, the foreign target sequence is specifically cut, and the foreign DNA can then be degraded by further cellular nucleases. Only the appropriate combination of PAM sequence and target sequence in appropriate spatial proximity leads to the cutting activity of the Cas nuclease. Since a specific combination of PAM and target sequence is generally not present in the genome of bacteria, an autoimmune reaction, i.e., the cutting of endogenous DNA, is prevented.

There are different CRISPR-Cas systems, differing especially in the nature and number of proteins required in order for the CRISPR complex to be able to bind and cut at the correct site in the target sequence. The systems that are particularly well understood are the type II-A systems, which, for the interference with the target DNA, use the Cas9 protein as effector nuclease. In addition to the eponymous CRISPR array which encodes the various crRNAs, the type II systems include a trans-activating RNA (tracrRNA), the adaptation proteins Cas1, Cas2 and Cas4, and the effector nuclease Cas9. The tracrRNA forms a hybrid with the crRNA encoded by the CRISPR array. Said hybrid, in turn, is specifically bound by Cas9. The crRNA portion of the hybrid construct directs Cas9 to the target DNA. The two-part system of crRNA and tracrRNA is relatively cumbersome for use in molecular biology, and so the crRNA and the tacrRNA are often linked by a linker. This resultant RNA is called sgRNA (single guide RNA).

It is an object of the invention to provide a simplified or improved method for linearizing circular DNA molecules that can be used as DNA templates for enzymatic RNA synthesis. To this end, the linearized DNA molecule shall have blunt ends and shall have a poly T sequence at the 5′ end of the template strand. In particular, the method shall provide linearized DNA molecules that allow the synthesis of RNAs having a homogeneous and uncovered poly A tail. It is a further object of the invention to provide a method that requires fewer method steps than the methods disclosed in the prior art. It is a further object of the invention to provide a method that yields linear DNA molecules having improved homogeneity in terms of the length of the poly A tail. It is a further object of the invention to provide a method that can be carried out more efficiently or more cost-effectively than the methods described in the prior art.

The object is achieved by a method for producing a linear double-stranded DNA molecule, the linear DNA molecule having blunt ends, and the linear DNA molecule having a poly T sequence at the 5′ end of the template strand, comprising the steps of:
(1) providing a double-stranded circular DNA molecule comprising on the template strand in the 3′ to 5′ direction:
(i) a template sequence operatively linked to an RNA polymerase promoter,
(ii) a poly T sequence,
(iii) a nucleotide sequence described by N₁N₂N₃, where N₁ to N₃ are each independently G, T, A or C or a nucleotide analog thereof, and
(iv) a protospacer adjacent motif (PAM),
(2) providing a guide RNA comprising a region which is complementary to the 5′ end of the poly T sequence (ii) and to the nucleotide sequence (iii) and a region which interacts with a Cas nuclease,
(3) providing a type II Cas nuclease,
(4) contacting the double-stranded circular DNA molecule with the guide RNA and the CAS nuclease.

The application of CRISPR-Cas systems in the field of linearization of plasmid DNA, in particular as template for in vitro transcription of RNA, is not yet known. Through a combination of a guide RNA, a Cas nuclease, for example Cas9, and a double-stranded circular DNA molecule, a CRISPR-Cas system can be used for the efficient and homogeneous linearization of plasmid DNA.

Owing to the possibility of flexible design of the guide RNA, the method for linearization can be used flexibly for all double-stranded circular DNA molecules, with a specific cut at a pre-selected site of the DNA being possible.

Owing to the possibility of flexible design of the PAM, the method for linearization can be carried out flexibly using different Cas nucleases.

On cutting the double strand in the target molecule, type II CAS nucleases, for example Cas9, form so-called “blunt” DNA ends, i.e., DNA ends without a 3′ or 5′ overhang. Such DNA templates having blunt ends are particularly highly suited to RNA synthesis. In mRNA synthesis, it is desirable for the resultant mRNA to have a homogeneous and uncovered poly A tail of well-defined length. The more homogeneous the poly A tails of the mature mRNA, i.e., the fewer the non-adenine bases, the easier and more efficient the binding of the poly A-binding protein. The binding of this protein in turn is crucial for effective translation of the mature mRNA within the target cell. Adverse effects of mRNAs which do not end directly with adenine residues or end with different bases, as arise for example when using many type II restriction enzymes, can thus be avoided. Thus the method according to the invention allows the generation of linear DNA templates which are particularly highly suited to producing a natural, mature RNA, for example an mRNA, which has advantages in therapeutic applications, for example improved stability, relatively long half-life or relatively high translation efficiency.

The method according to the invention offers a high degree of freedom in sequence design and absolute sequence independence, i.e., there are no constraints for nucleotides and for sequence and structure motifs.

The CRISPR-Cas-based method according to the invention is independent of methylations and other DNA modifications, as is the case with many restriction enzymes.

According to the invention, linearization of the double-stranded circular DNA molecule, i.e., the DNA template, can already occur during the replication of the plasmid DNA, i.e., for example within the bacterial production cell or before cell disruption. This has the advantage that the number of process steps can be reduced. Thus, at the end of pDNA fermentation, linearization by a Cas nuclease, for example Cas9, can already occur in the cell. Following completion of fermentation and intracellular linearization, the linear DNA can be purified by conventional methods; there is no need for separate processing of the double-stranded circular DNA molecule. Thereafter, the linear DNA template can be used directly in in vitro transcription for production of RNA, for example mRNA having an uncovered poly A tail.

The linear double-stranded DNA molecule produced according to the invention has blunt ends and a poly T sequence at the 5′ end of the template strand. Such linear DNA molecules are particularly highly suited as DNA template for in vitro transcription. In vitro transcription is an enzymatic catalysis in which an RNA is produced in vitro, this involving use of a DNA-dependent RNA polymerase to synthesize, on the basis of a DNA template, the RNA complementary to said template. The synthesis additionally requires ribonucleotides (NTPs), a buffer system, for example TRIS-HCl, magnesium ions, for example MgCl₂, and optionally an RNase inhibitor, an inorganic pyrophosphatase and spermidine. Monomeric RNA polymerases, which are generally bacteriophage enzymes, are typically used. Examples of the most commonly used polymerases are T7 RNA polymerase (UniProt: P00573) or T3 RNA polymerase (UniProt: Q778M8).

### DEFINITIONS

Unless otherwise defined, the technical and scientific terms used herein have the same meaning as usually understood by a person skilled in the art in the field of the present invention. Any technical feature mentioned in the following definitions may be applied to any embodiment of the invention.

The terms “nucleic acid sequence”, “nucleotide sequence”, “DNA sequence” and “RNA sequence” are known to a person skilled in the art and refer to an individual and specific succession of nucleotides.

The terms “polynucleotide” and “nucleic acid” are used interchangeably herein and refer to a succession of nucleotides of any length, either ribonucleotides or deoxyribonucleotides. These terms include single-stranded and double-stranded DNA or RNA, genomic DNA, cDNA, mRNA, saRNA, gRNA, siRNA, miRNA or circRNA, which can include purine and pyrimidine bases, nucleotide analogs or other naturally, chemically or biochemically modified, non-natural or derivatized nucleotide bases.

A nucleotide analog refers to a chemical compound structurally and functionally similar to the nucleotide, i.e., the nucleotide analog can be recognized as substrate by a polymerase. Nucleotide analogs are understood herein to mean in particular natively and non-natively occurring variants of the naturally occurring nucleotides adenosine, cytosine, thymidine, guanosine and uridine, for example chemically derivatized nucleotides having non-natively occurring functional groups which are added to or removed from the naturally occurring nucleotide or which replace the naturally occurring functional groups of a nucleotide. Examples of nucleotide analogs include N1-methylpseudouridine, 5-methoxyuridine, 5-methylcytidine, pseudouridine, N4-acetylcytidine and N6-methyladenine.

A poly T sequence as used herein is to be understood to mean a succession of thymine nucleotides in which other nucleotides, i.e., adenine, guanine or cytosine, are preferably absent.

A poly A sequence as used herein is to be understood to mean a succession of adenine nucleotides in which other nucleotides, i.e., thymine, guanine or cytosine, are preferably absent.

A poly T sequence or a poly A sequence may be segmented into two or more different segments, each having at least about 40 thymine or adenine nucleotides. The individual segments may each be interrupted by sequences of about 10-20 nucleotide in length that are not poly T or poly A sequences and can also contain G, C and U. Such segmentation can bring about improved stability of the poly T sequence or poly A sequence.

An uncovered poly A tail of an RNA is to be understood herein to mean a terminal poly A sequence in which the succession of adenine nucleotides at the 3′ end of the RNA is not followed by other nucleotides with the exception of adenine, i.e., not followed by cytosine, uracil or guanine.

A poly A tail of an RNA from an in vitro transcription is referred to herein as homogeneous if, in addition to the uncovered 3′ end, it has a narrow size distribution around a defined target length. Preferably, the average deviation from the target length is not more than 10%, based on the number of nucleotides, more preferably not more than 5% and particularly preferably less than 5%. A segmented poly A tail is considered homogeneous if the entirety of all the segments together has the abovementioned average deviation from the target length of the entirety of the segments. Segmentation can bring about stabilization of the entire poly A tail. In the case too of a segmented poly A tail of an RNA, it is advantageous that the 3′ segment of the poly A tail is uncovered to ensure high translation efficiency.

A template strand of a double-stranded DNA is understood to mean the DNA strand that is read by an RNA polymerase during transcription in order to synthesize the RNA.

A template sequence or template DNA, or a DNA template, is understood to mean the DNA sequence used for in vitro transcription. Said sequence serves as template for a DNA-dependent RNA polymerase in order to synthesize an RNA according to said sequence.

A template sequence may comprise a nucleic acid encoding an antigen, for example a tumor antigen, a viral antigen or a bacterial antigen, a therapeutic protein, or other proteins such as growth factors or transcription factors.

As used herein, a promoter sequence or promoter is a DNA regulatory region capable of binding an RNA polymerase and triggering the transcription of a downstream (in the 3′ direction) coding or noncoding sequence. Suitable promoters may be derived from any organism, including prokaryotic and eukaryotic organisms.

As used herein, operatively linked to a promoter means that the promoter brings about or regulates the transcription of a DNA containing the code of a gene, for example the gene whose code is specified by the template sequence or the gene encoding the guide RNA or the gene encoding a type II Cas nuclease. The promoter may be a native promoter, i.e., a promoter present in the cell into which the vector is introduced. In one embodiment, the promoter is an inducible promoter, i.e., the promoter is regulated in order to allow inducible transcription of a gene, such as the gene encoding the guide RNA or the gene encoding a type II Cas nuclease.

The promoter may be a constitutively active promoter, i.e., a promoter which is constitutively in an active state, or it may be an inducible promoter, i.e., a promoter, the state of which is controlled by an external stimulus or brought from the inactive state to an active state. A stimulus or inducer causes, i.e., activates, the expression of a target gene. This method is preferably used if the expression or overexpression of a target gene has adverse effects on the production organism or on a process. Such an external stimulus may be, for example, a specific temperature, a compound or a protein.

Examples of inducible promoters include the Lac promoter, which is IPTG-inducible (Jacob and Monod, Volume 3, Issue 3, Journal of Molecular Biology, June 1961, Pages 318-356, 1961), the tetracycline-regulated promoter (Gossen et al. 1995 Science 268 (5218): 1766-9), the rhamnose-inducible promoter (Kelly et al., ACS Synth. Biol.; 2016, 5 Pages 1136-1145), and the arabinose promoter, which is arabinose-inducible (Guzman et al., J Bacteriol. 1995 July; 177 (14): 4121-30.).

The term “protospacer adjacent motif” or PAM describes a signal sequence of about 2 to 8 base pairs which is the specific recognition site for a Cas nuclease or for a multiprotein Cas effector complex (e.g., CASCADE or CMR) on the DNA to be cut. If there is no PAM signal sequence present, the foreign DNA is not cut. The length and base sequence of the PAM sequence differ depending on the CRISPR-Cas system and Cas nuclease and depending on the associated organism in which the Cas nuclease naturally occurs. For example, the PAM sequence associated with the Cas9 nuclease of Streptococcus pyogenes (SpCas9) is 5′-NGG-3′, where “N” is any nucleotide, i.e., it can be A, T, G or C, followed by two guanine nucleotides. Further species and the associated PAM sequences are known to a person skilled in the art and described in the prior art (Fonfara et al. Nucleic Acids Research, 2014, Vol. 42, No. 4 2577-2590).

A guide RNA, gRNA, is a specific RNA sequence to which a Cas nuclease can bind and which directs said Cas nuclease to the template DNA. The guide RNA hybridizes specifically to a specific target site of the template DNA, where the Cas nuclease cuts the template DNA. The guide RNA may be a two-part guide RNA and be a hybrid of crRNA and tracrRNA. Alternatively, the guide RNA may be an individual sgRNA (single guide RNA) in which the essential regions of the crRNA and tracrRNA are linked to each other to form a continuous chimeric RNA. An sgRNA mimics the hairpin structure of the crRNA::tracrRNA duplex and is also capable of binding a Cas nuclease and directing it to the target sequence on the template DNA. The guide RNA is designed on the basis of the sequence information for the template DNA to be cut.

Complementary or complementarity, as used herein, describes the relationship between two DNA or RNA nucleic acid regions which match each other and form hydrogen bonds with each other, i.e., hybridize with each other, on the basis of their nucleobases as described by Watson and Crick. Known in the prior art are, for example, standard Watson-Crick base pairings: adenine (A) pairs with thymidine (T), adenine (A) pairs with uracil (U), and guanine (G) pairs with cytosine (C).

Homologous genes or homologous sequences are to be understood herein to mean that the DNA sequences of said genes or sections of DNA are at least 70% identical, preferably at least 80% identical, preferably at least 90% identical and particularly preferably at least 95% identical. The degree of DNA identity is determined by the “nucleotide blast” program, which can be found at http://blast.ncbi.nlm.nih.gov/and is based on the blastn algorithm. The algorithm parameters used to align two or more nucleotide sequences were the default parameters. The default general parameters are: Max target sequences=100; Short queries=“Automatically adjust parameters for short input sequences”; Expect Threshold=10; Word size=28; Automatically adjust parameters for short input sequences=0. The corresponding default scoring parameters are: Match/Mismatch Scores=1, −2; Gap Costs=Linear.

A homologous amino acid sequence is to be understood as meaning a sequence that is at least 70%, preferably at least 80%, preferably at least 90% and particularly preferably at least 95% identical, each alteration in the homologous sequence being selected from insertion, addition, deletion and substitution of one or more amino acids. Protein sequences are compared using the “protein blast” program at http://blast.ncbi.nlm.nih.gov/. This program uses the blastp algorithm. The algorithm parameters used to align two or more protein sequences were the default parameters. The default general parameters are: Max target sequences=100; Short queries=“Automatically adjust parameters for short input sequences”; Expect Threshold=10; Word size=3; Automatically adjust parameters for short input sequences=0. The default scoring parameters are: Matrix=BLOSUM62; Gap Costs=Existence: 11 Extension: 1; Compositional adjustments=Conditional compositional score template adjustment.

### DETAILED DESCRIPTION OF THE INVENTION

In one aspect, the present invention provides a method for producing a linear double-stranded DNA molecule, the linear DNA molecule having blunt ends, and the linear DNA molecule having a poly T sequence at the 5′ end of the template strand, comprising the steps of:
(1) providing a double-stranded circular DNA molecule comprising on the template strand in the 3′ to 5′ direction:
(i) a template sequence operatively linked to an RNA polymerase promoter,
(ii) a poly T sequence,
(iii) a nucleotide sequence described by N₁N₂N₃, where N₁ to N₃ are each independently G, T, A or C or a nucleotide analog thereof, and
(iv) a protospacer adjacent motif (PAM),
(2) providing a guide RNA comprising a region which is complementary to the 5′ end of the poly T sequence (ii) and to the nucleotide sequence (iii) and a region which interacts with a Cas nuclease,
(3) providing a type II Cas nuclease,
(4) contacting the double-stranded circular DNA molecule with the guide RNA and the CAS nuclease.

Provided for the method according to the invention is a double-stranded, circular DNA molecule that has been specifically designed. The circular DNA molecule comprises, on the template strand in the 3′ to 5′ direction, a template sequence operatively linked to an RNA polymerase promoter, a poly T sequence, and then a nucleotide sequence described by N₁N₂N₃, where N₁ to N₃ are each independently G, T, A or C or a nucleotide analog thereof, and a protospacer adjacent motif (PAM).

The RNA polymerase promoter is preferably a recognition sequence for a DNA-dependent RNA polymerase, for example the T7 RNA polymerase promoter, the SP6 RNA polymerase promoter or the T3 RNA polymerase promoter.

The template sequence comprises, for example, a nucleic acid encoding an antigen, for example a tumor antigen, a viral antigen or a bacterial antigen. In another example, the template sequence comprises a nucleic acid encoding a therapeutic protein or some other protein such as a growth factor or transcription factor.

The poly T sequence on the template strand of the double-stranded circular DNA molecule consists, for example, of between about 40 and about 250 T nucleotides. Preferably, the poly T sequence consists of between about 100 and about 140 T nucleotides, and more preferably consists of about 120 T nucleotides.

In the nucleotide sequence described by N₁N₂N₃, N₁ to N₃ are each independently G, T, A or C or a nucleotide analog thereof. Preferably, N₁ to N₃ are each independently G, T, A or C. In a particularly preferred embodiment, N₁ to N₃ are each independently G, A or C.

Additionally present on the template strand of the double-stranded circular DNA molecule is a protospacer adjacent motif (PAM). Preferably, the PAM immediately follows the nucleotide sequence N₁N₂N₃.

The PAM may be any selected PAM, in accordance with the specific Cas nuclease to be used in the method according to the invention. For example, the PAM comprises a sequence recognized by a type II Cas nuclease. For example, the PAM is selected from NGG, NGGNG, NNAAAAW, NG, NNNNACA, NNNNGATT, GNNNONNA and NNAGNN, where N in each case is independently selected from G, C, A and T, and where the PAM sequences are shown in the 5′ to 3′ direction. In a preferred embodiment, the PAM comprises the sequence 5′-NGG-3′, where N is independently selected from G, C, A and T. In another preferred embodiment, the PAM has the sequence 5′-NGG-3′, where N is independently selected from G, C, A or T. In a further preferred embodiment, the PAM is recognized by a Cas9 nuclease from Streptococcus.

The target site at which the circular double-stranded DNA is to be cut is preferably 3 nucleotides from the PAM in the 5′ direction.

The guide RNA for the method according to the invention is designed such that it has a sequence at the 5′ end that is complementary to the template strand of the double-stranded circular DNA molecule. This complementarity region consists, for example, of about 20 nucleotides which are complementary to about 20 consecutive nucleotides of the template strand.

The guide RNA comprises a region which is complementary to the 5′ end of the poly T sequence (ii) and to the nucleotide sequence (iii). Preferably, the region of the guide RNA which is complementary to the 5′ end of the poly T sequence (ii) and to the nucleotide sequence (iii) has the sequence 3′-G(A)ₙ-N₄N₅N₆-5′. In this case, n is a number between 17 and 23 and preferably a number between 17 and 20, and more preferably n is equal to 17.

N₄ to N₆ are each independently G, U, A, C or a nucleotide analog thereof, preferably N₄ to N₆ are each independently G, U, C or a nucleotide analog, and particularly preferably N₄ to N₆ are each independently G, U or C.

The complementarity sequence within the guide RNA has, for example, the sequence 5′-GAAAAAAAAAAAAAAAAANNN-3′, where N in each case is independently any nucleotide. Preferably, the method is characterized in that the guide RNA is SEQ ID NO: 1.

The complementarity of the region in the guide RNA to the 5′ end of the poly T sequence (ii) and to the nucleotide sequence (iii) is at least 90%, for example 90%, 91%, 92%, 93%, 94%, 95%, 96%, 97%, 98%, 99% or 100%. Preferably, the complementarity is 98%, 99% or 100%, particularly preferably 100%.

The nucleotides N₄N₅N₆ of the guide RNA are complementary to the nucleotides N₁N₂N₃ from the nucleotide sequence (iii) from the template strand. This means that N₄ is complementary to N₁, N₅ is complementary to N₂, and N₆ is complementary to N₃. Preferably, the complementarity of the nucleotides N₄N₅N₆ and the nucleotides N₁N₂N₃ is 100%. Preferred complementary base pairings include, for example, G and C, and A and T or A and U. Complementary sequences are, for example, CAG for N₁N₂N₃ and GUC for N₄N₅N₆.

Furthermore, the guide RNA comprises a region which interacts with a Cas nuclease. Preferably, the guide RNA comprises a region which interacts with a type II Cas nuclease.

Furthermore, provided in the method according to the invention is a type II Cas nuclease. Any type II Cas nuclease may be used here. For example, a type II Cas nuclease from Streptococcus pyogenes (SpCas9; UniProt Q99ZW2), Streptococcus mutans (SmCas9; UniProt Q8DTE3), Streptococcus thermophilus (StCas9; UniProt G3ECR1), Staphylococcus aureus (SaCas9; UniProt J7RUA5), Francisella novicida (FnCas9; UniProt A0Q5Y3), Campylobacter jejuni (CjCas9; UniProt A0A698TVI8), Neisseria meningitids (MnCas9; UniProt C9X1G5), Pasteurella multocida (PmCas9; AddGene #68703; https://www.addgene.org/68703/) is provided. Preferably, a type II Cas nuclease from Streptococcus spec. is provided. More preferably, a type II Cas nuclease from Streptococcus pyogenes is provided. Most preferably, the type II Cas nuclease used is Cas9 from Streptococcus pyogenes.

Preferably, the type II Cas nuclease used is Cas9. For example, Cas9 is selected from Streptococcus pyogenes having SEQ ID NO:2 or a sequence at least 70% homologous thereto, preferably at least 80% homologous sequence, more preferably at least 90% homologous sequence, more preferably at least 95% homologous sequence and particularly preferably at least 98% homologous sequence; Staphylococcus aureus having SEQ ID NO:3 or a sequence at least 70% homologous thereto, preferably at least 80% homologous sequence, more preferably at least 90% homologous sequence, more preferably at least 95% homologous sequence and particularly preferably at least 98% homologous sequence, or Streptococcus thermophilus having SEQ ID NO:4 or a sequence at least 70% homologous thereto, preferably at least 80% homologous sequence, more preferably at least 90% homologous sequence, more preferably at least 95% homologous sequence and particularly preferably at least 98% homologous sequence.

In a preferred embodiment, the type II Cas nuclease is Cas9 from Streptococcus pyogenes having SEQ ID NO:2 or a sequence at least 70% homologous thereto, preferably at least 80% homologous sequence, more preferably at least 90% homologous sequence, more preferably at least 95% homologous sequence and particularly preferably at least 98% homologous sequence.

If the Cas nuclease used in the method according to the invention is Cas9 from S. pyogenes, then the PAM used is preferably 5′-NGG-3′. If the Cas nuclease used is Cas9 from S. mutans, then the PAM used is preferably 5′-NGG-3′. If the Cas nuclease used is Cas9 a variant from S. thermophilus, then the PAM used is preferably 5′-NGGNG-3′ or 5′-NNAAAAW-3′. If the Cas nuclease used is Cas9 from Francisella novicida, then the PAM used is preferably 5′-NG-3′. If the Cas nuclease used is Cas9 from Campylobacter jejuni, then the PAM used is preferably 5′-NNNNACA-3′. If the Cas nuclease used is Cas9 from Neisseria menigitidis, then the PAM used is preferably 5′-NNNNGATT-3′. If the Cas nuclease used is Cas9 from Pasteurella multocida, then the PAM used is preferably 5′-GNNNCNNA-3′, there being no intention to limit the present invention to these examples.

In a further preferred embodiment, the present invention provides a method for producing a linear double-stranded DNA molecule, the linear DNA molecule having blunt ends, and the linear DNA molecule having a poly T sequence at the 5′ end of the template strand, comprising the steps of:
(1) providing a double-stranded circular DNA molecule comprising on the template strand in the 3′ to 5′ direction:
(i) a template sequence operatively linked to an RNA polymerase promoter,
(ii) a poly T sequence, the poly T sequence consisting of about 120 T nucleotides,
(iii) a nucleotide sequence described by N₁N₂N₃, where N₁ to N₃ are each independently G, T, A or C or a nucleotide analog thereof, and
(iv) a PAM having the sequence 5′-NGG-3′, where N is independently selected from G, C, A and T thereof,
(2) providing a guide RNA comprising a region which is complementary to the 5′ end of the poly T sequence (ii) and to the nucleotide sequence (iii) and a region which interacts with a Cas nuclease, the region which is complementary to the 5′ end of the poly T sequence (ii) and to the nucleotide sequence (iii) having the sequence 3′-G (A)ₙN₄N₅N₆-5′, where n is equal to 17, where N₄ to N₆ are each independently G, U or C,
(3) providing a type II Cas nuclease, the type II Cas nuclease being Cas9 from Streptococcus pyogenes having SEQ ID NO:2 or a sequence at least 70% homologous thereto,
(4) contacting the double-stranded circular DNA molecule with the guide RNA and the CAS nuclease.

In the method according to the invention, the guide RNA may be provided as an RNA molecule. According to the invention, the providing of the guide RNA may also be achieved by the expression of a nucleotide sequence encoding the guide RNA. Preferably, the guide RNA is provided by the expression of a nucleotide sequence encoding the guide RNA.

In the method according to the invention, the type II Cas nuclease may be provided as a protein. According to the invention, the providing of the type II Cas nuclease may also be achieved by the expression of a nucleotide sequence encoding the type II Cas nuclease. Preferably, the type II Cas nuclease is provided by the expression of a nucleotide sequence encoding the type II Cas nuclease. Particularly preferably, both the guide RNA and the Cas nuclease are provided by the expression of a nucleotide sequence. The nucleic acid encoding the guide RNA and/or the Cas nuclease may be located on an expression plasmid. For example, the nucleic acid encoding the guide RNA may be located on one expression plasmid and the nucleic acid encoding the Cas nuclease may be located on a separate expression plasmid. In another example, the nucleic acid encoding the guide RNA and the nucleic acid encoding the Cas nuclease are located on the same expression plasmid.

For example, an expression plasmid comprising Cas9 and guide RNA may be generated as follows. With cloning, a Cas9 nuclease may first be cloned into a suitable expression plasmid, followed by, for example, cloning of a tracrRNA construct and a minimal CRISPR array in the 5′-3′ direction. This allows, for example, natural formation of a tracrRNA::crRNA duplex of a guide RNA. A suitable expression vector is, for example, pWAC003 (SEQ ID NO:5). This is a low-copy plasmid with approx. 10 copies per cell based on the pACYC origin of replication.

According to the invention, what occurs is contacting of the double-stranded circular DNA molecule with the guide RNA and the CAS nuclease. The contacting may occur, for example, in vitro. In an embodiment in which the providing of the guide RNA and/or the type II Cas nuclease is achieved by the expression of a nucleotide sequence encoding the guide RNA and/or the type II Cas nuclease, the contacting preferably occurs in a cell, in particular in a bacterial cell capable of multiplying plasmid DNA.

Constitutive expression of the Cas endonuclease, for example Cas9, can cause non-specific DNA cutting events at various sites in the plasmid product, i.e., in the double-stranded circular DNA molecule, or in the chromosome of the bacterial cell used. Early processing of the double-stranded circular DNA molecule by a Cas nuclease, for example Cas9, in the cells can thus lead to the degradation of the desired linear DNA product, since the free ends of a linear DNA are recognized by corresponding DNases. Moreover, the cell as producer can also be damaged. In order to reduce this effect, it is preferred to use a regulatable promoter having minimum basal expression for the expression of the type II Cas nuclease, for example Cas9.

In addition to this, for the inducible expression of the Cas nuclease, for example Cas9, preference is given to possible limitation of the derepression of the promoter, i.e., possible limitation of the inducible expression level. Excessively high expression of the Cas nuclease, for example Cas9, within a short period of time can have adverse effects on pDNA quality and the producer cell similar to those with high basal expression over a relatively long period of time. Therefore, particular preference is given to promoters which have low basal expression and, following induction, a moderate expression level.

The advantage of inducible expression of the guide RNA over constitutive expression is that the expression does cause constant stress on the producer cell. As a result of the lower stress, the cell is able to produce more of the actual target product, the circular DNA.

The use of a regulatable or inducible promoter allows the time of production of the Cas nuclease and the guide RNA to be chosen specifically through the induction of expression. Regulatable or inducible expression of the processing apparatus, i.e., the Cas9 nuclease and the guide RNA, within the cell allows sparing of individual steps in the subsequent process, which leads to economic advantages. For example, additional linearization steps or separate purification of the plasmid DNA may be omitted.

Preferably, the method according to the invention is characterized in that the expression of the type II Cas nuclease and/or the guide RNA is inducible. This means that the nucleotide sequence encoding the guide RNA and/or the nucleotide sequence encoding the Cas nuclease is/are operatively linked to an inducible promoter. Preferably, both the nucleotide sequence encoding the guide RNA and the nucleotide sequence encoding the Cas nuclease are operatively linked to an inducible promoter.

In one embodiment, the nucleotide sequence encoding the guide RNA and the nucleotide sequence encoding the Cas nuclease are operatively linked to the same inducible promoter. When an identical or similar induction system is used, the expression of the guide RNA and the Cas nuclease starts at the same time. Firstly, this procedure allows the lowering of cellular stress and, secondly, the synchronized start of expression allows a more appropriate ratio of the molecules, Cas endonuclease vs. guide RNA.

In another embodiment, the nucleotide sequence encoding the guide RNA and the nucleotide sequence encoding the Cas nuclease are operatively linked to different inducible promoters, for example for sequential expression.

Inducible promoters are known to a person skilled in the art. Examples of suitable inducible promoters are the lac, tac, trc, lambda PL, ara, cumate or tet promoter or sequences derived therefrom.

Preferably, according to the invention, the inducible promoter used is a lactose-inducible promoter, in particular the promoter WAC003 having the sequence SEQ ID NO: 6 or the promoter WAC003-LacIDel having the sequence SEQ ID NO:7.

In one embodiment, the nucleotide sequence encoding the guide RNA and/or the nucleotide sequence encoding the Cas nuclease has/have been integrated into the genome of E. coli. In a preferred embodiment, both the nucleotide sequence encoding the guide RNA and the nucleotide sequence encoding the Cas nuclease have been integrated into the genome of E. coli. In another preferred embodiment, the nucleotide sequence encoding the Cas nuclease has been integrated into the genome of E. coli, but not the nucleotide sequence encoding the guide RNA. Methods for integrating nucleotide sequences into bacterial genomes are known to a person skilled in the art (e.g., Datsenko et al. 2000, Proc. Natl. Acad. Sci. USA. 97:6640-5).

In another aspect, the present invention provides for the use of a type II Cas nuclease for linearization of a double-stranded, circular DNA molecule, the linear DNA molecule comprising on the template strand in the 3′ to 5′ direction:
(i) a template sequence operatively linked to an RNA polymerase promoter,
(ii) a poly T sequence,
(iii) a nucleotide sequence described by N₁N₂N₃, where N₁ to N₃ are each independently G, T, A or C or a nucleotide analog thereof, and
(iv) a protospacer adjacent motif (PAM).

In a further aspect, the present invention provides for the use of a recombinant microorganism in which the nucleotide sequence encoding a guide RNA and/or the nucleotide sequence encoding a type II Cas nuclease has/have been integrated into the genome for production of a linear double-stranded DNA molecule, the linear DNA molecule having blunt ends, and the linear DNA molecule having a poly T sequence at the 5′ end of the template strand.

One embodiment provides for the use of a recombinant microorganism in which the nucleotide sequence encoding a guide RNA and the nucleotide sequence encoding a type II Cas nuclease have been integrated into the genome. Another embodiment provides for the use of a recombinant microorganism in which the nucleotide sequence encoding a type II Cas nuclease has been integrated into the genome for production of a linear double-stranded DNA molecule.

The recombinant microorganism may be any microorganism suitable for the generation of plasmid DNA. Preferably the recombinant microorganism is E. coli, more preferably the recombinant microorganism is an E. coli K12 strain, and particularly preferably the recombinant microorganism is E. coli NEB10Beta.

The invention is further described in more detail hereinbelow with reference to exemplary embodiments and the accompanying figures, without being limited thereby.

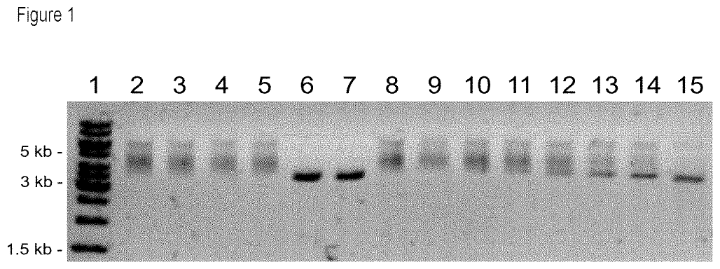

FIG. 1 shows an agarose gel containing a plasmid DNA resolved by gel electrophoresis and various controls: 1: marker (GeneRuler™ 1 kb DNA Ladder) 2: pGFP1 without incubation (negative control 1) 3: pGFP1 in water with incubation (negative control 2) 4: pGFP1 in cutting buffer (negative control 3) 5: pGFP1 in cutting buffer+1 mM EDTA (negative control 4) 6: pGFP1 incubated with XbaI (positive control 1) 7: pGFP1 incubated with NcoI (positive control 2) 8: pGFP1 incubated with 25 nM Cas9 (negative control 5) 9: pGFP1 incubated with 25 nM sgRNA (negative control 6) 10-15: test samples 1-5 (1-fold, 2-fold, 3-fold, 4-fold, 5-fold and 10-fold excess of Cas9/guide RNA in relation to pDNA).

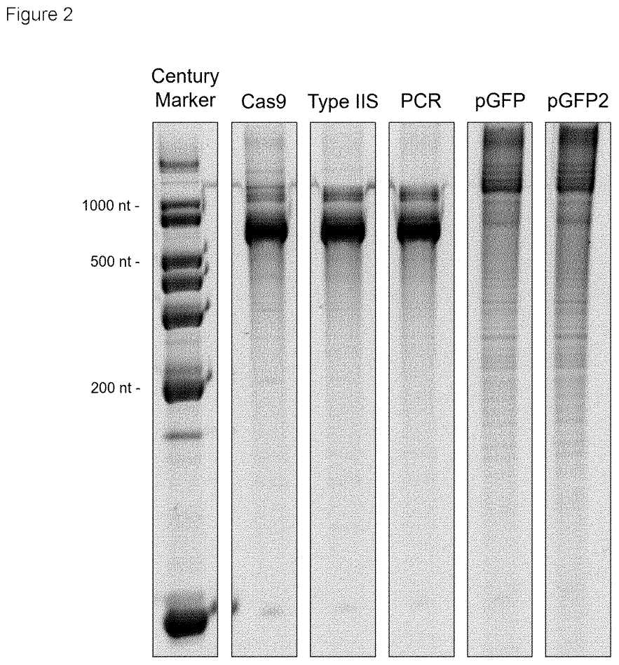

FIG. 2 shows an agarose gel containing mRNAs resolved by gel electrophoresis, said mRNAs having been formed in in vitro transcription using different DNA templates. Cas9 refers to the sample in which the DNA linearized by means of SpCas9/guide RNA was used as template DNA. Type IIS refers to the sample in which the DNA linearized by means of type IIS restriction enzyme was used as template DNA. PCR refers to the sample in which the DNA generated by PCR was used as template. pGFP and pGFP2 refer to the samples in which the circular plasmids pGFP1 and pGFP2 were used as DNA templates.

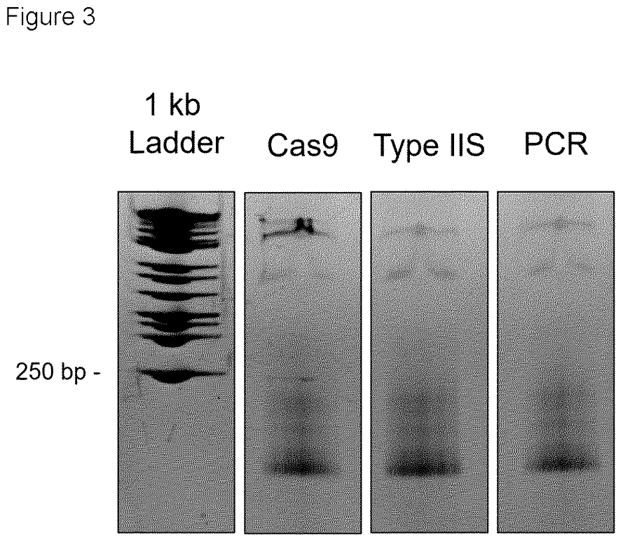

FIG. 3 shows the analysis of the poly A tail by acrylamide gel electrophoresis. 1: marker, GeneRuler™ 1 kb DNA Ladder (Thermo Fisher Scientific). Cas9: RT-PCR product for the RNA produced using a template DNA linearized by means of SpCas9. Type IIS: RT-PCR product for the RNA produced using a template DNA linearized by means of a type IIS restriction enzyme. PCT: RT-PCR product for the RNA produced using a template DNA produced by PCR.

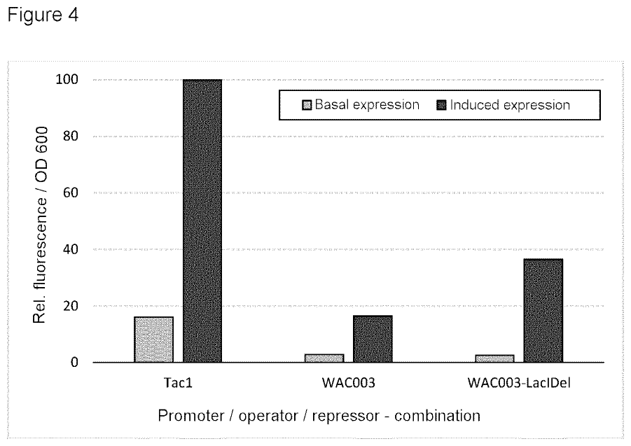

FIG. 4 shows the basal expression and expression level by the modified promoters WAC003 and WAC003-LacIDel compared to the pTac1 parent variant using the example of the expression of the reporter protein GFP in E. coli K12.

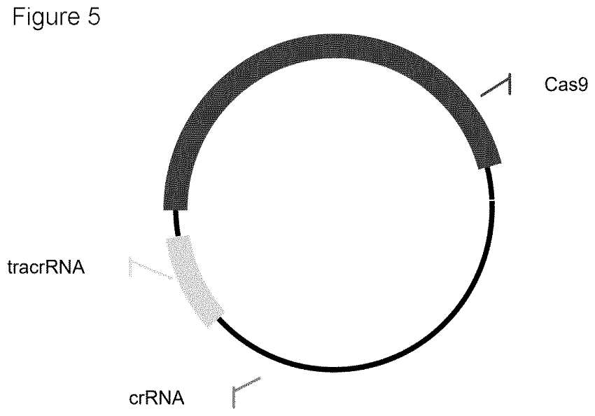

FIG. 5 shows a schematic illustration of the plasmid pCas9_tracrRNA_CRISPR.

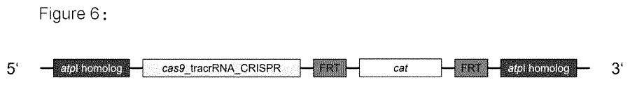

FIG. 6 is a schematic illustration of a construct for genomic integration by A Red recombination.

### EXAMPLES

### Example 1: Plasmid DNA-Cutting Activity by Cas9

To produce the plasmid, a construct comprising template sequence, poly A tail, and target sequence for cutting by a Cas nuclease, and PAM was first designed. Besides a GFP mRNA template sequence, said construct encodes at the 3′ end of said sequence a poly A tail, followed in turn by three further nucleotides (5′-GTC-3′) and the protospacer adjacent motif (PAM) 5′-CGG-3′. The PAM sequence is the Cas9 gRNA recognition sequence for processing. To create the template DNA sequence, the gfp gene (UniProt: C5MKY7) was amplified using the two primers (SEQ ID NO:8 and 9) appropriate for it with the Phusion polymerase (Thermo Fisher Scientific) in a PCR according to the standard protocol. The two primers comprised, firstly, the T7 RNA polymerase promoter (forward primer, SEQ ID NO:8) and, secondly, the elements: poly T, 5′ GAC 3′ and PAM (5′-CGG-3′) (reverse primer, SEQ ID NO:9). The PCR product (SEQ ID No:10) was then cloned into a pUC18 vector (New England Biolabs) via the XbaI and EcoRI cleavage sites. The resultant plasmid pGFP1 (SEQ ID NO:11) was used in the following experiments/examples as starting plasmid for the production of the linearized template DNA.

After the plasmid pGFP1 had been successfully produced, the plasmid DNA was produced with the aid of E. coli NEB10Beta (New England Biolabs). This was achieved by transforming the NEB10Beta cells with the plasmid pGFP1 using a heat-shock method according to the manufacturer's instructions. To this end, the cells and 100 ng of pGFP1 were incubated on ice for 30 min, followed by a 30-second heat shock at 42 °C. After a regeneration phase (1 h at 37 °C), the transformants were selected on selection medium (LB-ampicillin (LB-Amp)). Clones formed were grown in 3 mL cultures under selective conditions and the plasmid of the clones was verified via restriction patterns and sequencing. Clones having the desired plasmid were grown in 50 ml of LB-Amp (shaking, 37 °C overnight) to generate sufficient amounts of plasmid DNA. Depending on the clone, an OD₆₀₀ (optical density at 600 nm against water) of 8-14 in a shake flask was achieved. The plasmid DNA was lastly isolated and purified from the overnight culture (stationary phase reached) using the Gene Jet Plasmid Kit (Thermo Fisher Scientific) according to the manufacturer's instructions.

In a next step, a template DNA for the in vitro production of a guide RNA (SEQ ID NO:1) was designed. To allow the synthesis of a guide RNA of specific length and specific sequence, the DNA sequence complementary to the guide RNA was added 3′ downstream of a T7 RNA polymerase promoter sequence (SEQ ID NO:12). Together with the complementary sequence of the T7 promoter (SEQ ID NO:13), it was synthesized as long single strands (primers) (Metabion).

To produce the guide RNA (SEQ ID NO:1), these two DNA oligonucleotides (SEQ ID NO: 12 and 13) were hybridized with each other by mixing the two oligonucleotides in equimolar amounts (100 μmol), boiling them at 95 °C for 5 min and then slowly cooling them down to room temperature. The hybrid generated was then used as template to synthesize the guide RNA using an in vitro transcription kit (HighYield T7 RNA Synthesis Kit from Jena Bioscience) according to the manufacturer's instructions. Thereafter, the transcription reaction was purified using the Monarch RNA Cleanup Kit from New England Biolabs (NEB) according to the manufacturer's instructions and the concentration determined by UV absorption.

After the plasmid pGFP1 (SEQ ID NO:11) and the guide RNA (SEQ ID NO:1) had been produced, the two were incubated together with the Cas nuclease SpCas9 (New England Biolabs) in a cutting buffer (20 mM Hepes pH 7.5; 150 mM KCl, 10 mM MgCl₂, 0.5 mM DTT, 0.1 mM EDTA). SpCas9 and the guide RNA were used equimolarly and both were used in varying excess in relation to the pDNA (5 nM) (1-fold, 2-fold, 3-fold, 4-fold, 5-fold and 10-fold). For better complex formation, SpCas9 and the guide RNA were first incubated on their own (15 min, 37 °C). The pDNA (5 nM) and the reaction buffer were then added. After an incubation time of 60 min at 37 °C, the reaction was stopped by addition of 20 μL of 2× formamide buffer (Thermo Fisher Scientific) and the pDNA was analyzed by agarose gel electrophoresis (FIG. 1).

For the analysis, the cut pDNA was separated across a 1% agarose gel (TAE-buffered) and then visualized using Midori Green (Nippon Genetics Europe) on a gel documentation system (BioRad) (see FIG. 1). The controls used were uncut and nonincubated plasmid DNA, uncut incubated (1 h, 37 °C) pDNA, pDNA with cutting buffer, pDNA with cutting buffer and 1 mM EDTA, pDNA with 25 nM Cas9 without guide RNA, pDNA with 25 nM guide RNA without Cas9, and, as positive control, pDNA (SEQ ID NO: 11) linearized by means of XbaI or NcoI (used according to the manufacturer's instructions, Thermo Fisher Scientific).

FIG. 1 shows that the plasmid DNA pGFP1 is linearized by the Cas9/guide RNA complex. Loaded in lane 1 was the marker GeneRuler™ 1 kb DNA Ladder (Thermo Fisher Scientific). The controls used were: pGFP1 without incubation (lane 2, starting material), pGFP1 in water with incubation (lane 3, without Cas9 and guide RNA), pGFP1 in cutting buffer (lane 4, without Cas9 and guide RNA), pGFP1 in cutting buffer+1 mM EDTA (lane 5, without Cas9 and guide RNA), pGFP1 incubated with XbaI (lane 6), pGFP1 incubated with NcoI (lane 7), pGFP1 incubated with 25 nM Cas9 (without guide RNA; lane 8), pGFP1 incubated with 25 nM guide RNA (without Cas9; lane 9). The test samples were loaded in lanes 10-15 (1-fold, 2-fold, 3-fold, 4-fold, 5-fold and 10-fold excess of Cas9/guide RNA in relation to pDNA). FIG. 1 additionally shows that, as the amount of Cas9/guide RNA complex increases, more pDNA is linearized. In the case of a 10-fold molar excess (lane 15), the degree of linearization achieved is virtually the same as that achieved with the use of conventional type II restriction enzymes. This is shown exemplarily by the reactions using XbaI and NcoI (lanes 6 & 7). The dependence on Cas9/guide RNA complex formation is shown by the two lanes 8 and 9, in which one component is omitted in each case, either the guide RNA (lane 8) or Cas9 (lane 9), under a 10-fold molar excess. No linearization can be observed here, as is the case with pGFP1 samples in which no enzymes were used (lane 3, lane 4, lane 5).

### Example 2: MRNA Synthesis from pDNA Cut by Cas9

The plasmid DNA pGFP1 (SEQ ID NO:11) was linearized as described in Example 1 by means of SpCas9 and the guide RNA (SEQ ID NO:1) (10-fold molar excess; see also FIG. 1, lane 15). The linearized plasmid was then purified using the Gene Jet PCR Purification Kit (Thermo Fisher Scientific). The linear, purified pDNA was then used as template DNA in an in vitro transcription reaction (HighYield T7 RNA Synthesis Kit, Jena Bioscience) according to the manufacturer's instructions to synthesize an mRNA. The reaction was carried out at 37 °C for 2 h using 200 ng of template DNA. To remove impurities and kit components, for example enzymes and nucleotides, from the reaction, the mRNA was purified using a Monarch RNA Cleanup Kit (NEB) according to the manufacturer's instructions.

For comparison of mRNA production processes using the method according to the invention with the methods from the prior art, i.e., linearization of a plasmid DNA by means of type IIS restriction enzymes or production of a PCR product as linear starting DNA, pGFP2 (SEQ ID NO:14, produced in analogy to pGFP1, by amplification of the gfp gene by means of primers of SEQ ID NO:9 and 15) was treated and linearized by means of the type IIS restriction enzyme BpiI according to the manufacturer's instructions (Thermo Fisher Scientific). In addition, a linear starting DNA was produced by means of a polymerase chain reaction (PCR product) (SEQ ID NO:16). In this case, use was made of a primer having a T7 RNA polymerase promoter (forward primer, SEQ ID NO: 17) together with a primer having a 60 nt poly A tail (reverse primer, SEQ ID NO: 18) and with pGFP1 as PCR template (10 ng) and the Phusion DNA polymerase. The reaction was carried out in the supplied buffer system according to the manufacturer's instructions (Thermo Fisher Scientific). Following the reaction, the PCR product was digested with DpnI to remove any remaining methylated plasmid DNA from the PCR reaction. Lastly, both the PCR product and the type IIS-linearized plasmid were purified using a PCR Purification Kit (Thermo Fisher Scientific) according to the manufacturer's instructions. Whereas the linear DNA from the PCR reaction and the SpCas9-linearized DNA each had blunt ends, the type IIS-linearized DNA had a 3′ overhang.

The linear DNAs from the restriction digest and from the PCR reaction were used in the same amounts as the SpCas9-linearized pDNA (200 ng) in an in vitro transcription reaction as template DNA for synthesis of an mRNA according to the manufacturer's instructions (HighYield T7 RNA Synthesis Kit, Jena Bioscience). The synthesis was carried out at 37 °C for 2 h. Lastly, the mRNAs were purified using the Monarch RNA Cleanup Kit (NEB) according to the manufacturer's instructions.

The mRNAs produced were first analyzed by polyacrylamide gel electrophoresis. In this case, 200 ng of mRNA from each reaction were admixed with RNA loading buffer (Gel Loading Buffer 2, Thermo Fisher Scientific) and boiled at 95 °C for 5 min. The RNA species were then analyzed by resolution on a 15% TBE-UREA polyacrylamide gel (Thermo Fisher Scientific; 250 V, 50 min). Following electrophoresis, the gel was stained using SYBR Green II RNA Gel Stain (Thermo Fisher Scientific) and visualized in a gel documentation system (BioRad) (FIG. 2). The controls used were in vitro transcription reactions with 200 ng of uncut pGFP1 and pGFP2. pGFP2 is a pGFP1 variant lacking a PAM sequence (SEQ ID NO:14). The concentration of the mRNAs produced was determined photometrically using a NanoDrop photospectrometer (Thermo Fisher Scientific).

FIG. 2 is a visualization of the mRNAs produced. Lane 1 contains the Century Marker™ (Thermo Fisher Scientific) and the following lanes contain the RNA samples corresponding to the starting DNAs: SpCas9/guide RNA-linearized DNA (Cas9), type IIS restriction enzyme-linearized DNA (Type IIS), DNA from the PCR reaction (PCR), and the uncut plasmids pGFP1 and pGFP2. FIG. 2 clearly shows that the main mRNA products obtained from the reactions with linearized plasmids (Cas9, Type IIS) and PCR product (PCR) have identical sizes. Only in reactions with nonlinearized plasmids (pGFP1 and pGFP2) were larger products apparent. These products result from the “unchecked” in vitro transcription by the T7 RNA polymerase. The lack of linearization means that “run-off” of the polymerase is not possible, which leads to “read-through” and thus to heterogeneous and relatively large products.

To analyze the poly A tail of the mRNAs, an adapter oligonucleotide (SEQ ID NO:19) was ligated to the 3′ end of the mRNA by means of a T4 RNA ligase 1 (NEB). T4 RNA ligase 1 is an enzyme which can ligate single-stranded RNA. Said enzyme ligates a 5′-monophosphate-containing substrate to a 3′-OH end of the recipient mRNA. The 3′-OH end is naturally present in the RNA produced. The adapter oligonucleotide was synthesized to contain a 5′-monophosphate (Metabion). To prevent self-ligation of the adapter oligonucleotides, the 3′ end of the adapter oligonucleotide was also provided with a synthetic phosphate residue. For the ligation, 5 μg of RNA and 40 μmol of the adapter oligonucleotide were used. The reaction (50 mM Tris-HCl PH 7.5, 10 mM MgCl₂, 1 mM DTT, 0.5 U RNase inhibitor, 1 mM ATP, 10 U T4 RNA ligase) was incubated at 25 °C for 2 h. Then the buffer was exchanged for water by means of an Amicon (Merck) (100 kDa cut-off).

The adapter ligation products obtained (adapter oligonucleotide+mRNA) were subsequently amplified by means of a reverse transcriptase reaction (RT-PCR). For the RT-PCR, use was made of the Superscript™ Kit according to the manufacturer's instructions (Thermo Fisher Scientific). For the reaction, use was made of specific primers which directly bind 5′ upstream of the poly A (forward, SEQ ID NO:20) and to the adapter sequence (reverse, SEQ ID NO:21). The products present from the RT-PCR were purified using a PCR Purification Kit from Thermo Fisher according to the manufacturer's instructions and the samples were then analyzed by electrophoresis in a 15% polyacrylamide gel. For this purpose, the resolved DNA was stained using Midori Green (Biozym) and visualized in a gel documentation system (BioRad) (see FIG. 3).

FIG. 3 shows the analysis of the poly A tail by acrylamide gel electrophoresis. Loaded in lane 1 was the marker, GeneRuler™ 1 kb DNA Ladder (Thermo Fisher Scientific). Resolved in the following 3 lanes were the RT-PCR products for the RNAs produced using DNA templates linearized in different ways (SpCas9-linearized (lane 2), type IIS restriction enzyme-linearized (lane 3) and PCR product (lane 4)).

It can be seen in FIG. 3 that the same products with exactly the same size distribution of RT-PCR products were obtained in all 3 RNA samples. Since the source RNAs already have very large similarities (cf. FIG. 2), it can be assumed that the RT-PCR here had the same templates or very similar templates as starting material. The gel patterns thus show that the method according to the invention does not just allow effective linearization of pDNA, but also that the pDNA is linearized at the correct, predefined site and the desired uncovered free poly A tail is formed.

### Example 3: Promoter Design for Inducible Expression of Cas9

Since constitutive expression of a Cas endonuclease, for example Cas9, in a bacterial cell in vivo can cause nonspecific DNA cutting events at various sites both in plasmids and in the chromosome of a bacterial cell and can thus damage the bacterial cell as plasmid producer, preference is given to inducible expression of the Cas endonuclease, for example Cas9, under a regulatable promoter having minimum basal expression.

For the inducible expression of the Cas nuclease and hence the in vivo linearization of the plasmid product “activatable” by addition of inducer at a desired cultivation time, preference is additionally given to limitation of the inducible expression level. Excessively high expression of a Cas nuclease within a short period of time can have adverse effects on the plasmid DNA and the producer cell similar to those with high basal expression over a relatively long period of time. Therefore, particular preference is given to promoters which have both low basal expression and, following induction, a moderate expression level.

The pTac1 promoter (de Boehr, 1983, Proceedings of the National Academy of Sciences 80 (1): 21-5), a hybrid of the trp and lac promoters, has from the original Lac operon only one remaining operator site LacO1 at position +1 (transcription start site) for binding of the repressor protein LacI. The repressor protein LacI itself, consisting of an N-terminal “headpiece” DNA-binding unit, a hinge region, a core region having N-terminal and C-terminal subdomains including a lactose-binding region and a dimerization motif, and a C-terminal mini-zipper for tetramerization, associates to form a dimer of dimers. Each LacI dimer binds a 21 bp duplex DNA operator sequence LacO (Lewis 2011. J Mol Biol. 409, 14-27). In the native Lac operon, two duplex DNA operator sites (LacO1 and LacO3) are simultaneously bound by the associated LacI tetramer, which forms a so-called “repression loop” in the DNA strand for maximum repression (Oehler et al. 2006, Nucleic Acids Res. 34, 606-612).

In WAC003 (SEQ ID NO:6) and WAC003-LacIdel (SEQ ID NO:7), a synthetic LacO binding site was inserted via the −35 region of the pTac1 promoter without altering the nucleotide sequences of the −35 and −10 promoter regions, the distance (spacer) between the two regions, or the nucleotide sequence of the spacer itself. WAC003 interacts with the native, i.e., tetramerization-capable, LacI repressor protein. In the construct WAC003-LacIdel, the lacI gene was replaced by a variant LacIdel which, as a result of a nucleotide frameshift at the C-terminus of the encoded LacI protein, forms a different amino acid sequence and thus can no longer form the LacI tetramer, but only the LacI dimer.

Basal expression (repression) and the expression achievable after addition of inducer (derepression) were examined using the reporter protein GFP. GFP, as a cytoplasm-localized protein, does not itself stress the cells and is therefore highly suited as reference protein for measurement of expression level. The gene for GFP was placed under the control of the WAC003 and WAC003-LacIdel promoter constructs and cloned onto a plasmid having a ColE1 origin of replication, the plasmids were transformed into an E. coli K12 strain, and basal expression and inducible expression in a shake culture in LB medium were tested as follows.

From precultures, main cultures were inoculated to OD₆₀₀=0.1. After 2 h, with correlating cell densities of OD₆₀₀~0.4, 0.1 mM IPTG was added (=induction) or this did not occur (=basal expression). After a further 24 h of cultivation, the amount of GFP expressed was measured (excitation 380 nm, emission 500 nm) and normalized to the respective underlying cell density OD₆₀₀ of the culture. The control included was a cell line having the pTac1 promoter (high basal expression, high inducible expression level).

FIG. 4 shows the basal expression and expression level of the WAC003 and WAC003-LacIdel promoter constructs compared to the Tac1 parent variant. The WAC003 variant showed distinctly reduced basal expression compared to Tac1. After addition of IPTG, the derepression of the WAC003 promoter was likewise distinctly reduced (only ~16% GFP/OD₆₀₀ compared to Tac1). In the WAC003-LacIdel construct, basal expression is likewise low, and although the inducible expression level is somewhat higher than in WAC003, it is still very low compared to Tac1. The promoters WAC003 and WAC003-LacIdel are therefore very highly suited to the inducible expression of a Cas nuclease, for example Cas9, which is to be expressed in vivo for specific linearization of plasmid DNA.

### Example 4: In Vivo Induction of Cas9 and Purification of Linear DNA after Incubation

For analysis of the in vivo functionality of the system described in Example 1 and Example 2, both the gene encoding SpCas9 (SEQ ID NO:2) and the construct for the guide RNA (SEQ ID NO:1) were cloned in the same plasmid (pWAC003, SEQ ID NO:5), both under the inducible promoter WAC003-LacIDel (SEQ ID NO:7). Two SpCas9-specific primers (SEQ ID NO:22 and 23) were used to amplify the Sp_cas9 gene from the plasmid pCas9-ts1 (SEQ ID NO:24) for cloning. The resultant PCR product was cloned into the plasmid pWAC003 (SEQ ID NO:5) after the promoter WAC003-LacIDel (SEQ ID NO:7) using EcoRI and XbaI. The Cas9 expression cassette of the resultant plasmid pCas9 (SEQ ID NO:25) was sequenced using specific primers (SEQ ID NO:26 and SEQ ID NO:27) (Sequiserve) in order to confirm the desired sequence.

Since, in an in vivo system, the guide RNA cannot be produced by run-off transcription, as described in Example 2, adaptation of the RNA design is required. To generate a functional guide RNA, use must be made of the natural form of RNA processing by CRISPR-Cas9 systems. This means providing a minimal CRISPR array and a tracrRNA.

Inserted into the plasmid pCas9 (SEQ ID NO:25) were a tracrRNA sequence (SEQ ID NO: 28) and a CRISPR sequence (SEQ ID NO:29). In addition to the tracrRNA sequence and the CRISPR sequence, the sequences SEQ ID NO:28 and SEQ ID NO:29 each comprise a WAC003-LacIdel promoter sequence and a Rho-independent terminator sequence in which the tracrRNA and CRISPR sequence are embedded (5′-3′). For this purpose, the two DNA fragments were each first produced by PCR; the primers used for SEQ ID NO:28 were those having SEQ ID NO:30-35 and the primers used for SEQ ID NO: 29 were those having SEQ ID NO:36-41. The tracrRNA sequence was cloned into pCas9 using the two restriction enzymes KpnI and XbaI, such that that said sequence is 3′ downstream of the Cas9 construct. The CRISPR sequence was cloned into the resultant plasmid pCas9_tracrRNA (SEQ ID NO:42) in the 3′ position after the tracrRNA sequence via the XmaI and XbaI cleavage sites. The correct assembly of the resultant plasmid pCas9_tracrRNA_CRISPR (SEQ ID NO:43) was verified by sequencing using the specific primer (SEQ ID NO:27).

FIG. 4 shows a schematic illustration of the plasmid pCas9_tracrRNA_CRISPR. In the plasmid pCas9_tracrRNA_CRISPR, all three elements Cas9, tracrRNA and CRISPR are each under the control of a WAC003-LacIdel promoter and additionally have a Rho-independent terminator sequence.

The plasmid pCas9_tracrRNA_CRISPR (SEQ ID NO:43), together with pGFP1 (SEQ ID NO: 11), was transformed into E. coli NEB10Beta. After selection on LB-ampicillin/tetracycline, clones were inoculated in 20 mL of LB containing antibiotics (100 mg/L ampicillin or 20 mg/L tetracycline) and incubated overnight at 37 °C (200 rpm). The overnight cultures were used to inoculate 100 ml of fresh LB medium containing ampicillin and tetracycline (100 mg/L and 20 mg/L, respectively). The resultant main culture was incubated at 37 °C with shaking up to an OD₆₀₀ₙₘ of 0.8. Once the target OD was reached, IPTG was added to a final concentration of 0.5 mM for simultaneous induction of the expression of the Cas9, CRISPR cassette and tracrRNA. Owing to the coexpression of these elements, the plasmid pGFP1 is specifically cleaved. 2 h after induction, the cells were sedimented by centrifugation. The DNA in the supernatant (linearized pGFP1 and circular pCas9_tracrRNA_crRNA) was subsequently isolated and purified using the Gene Jet Plasmid Kit (Thermo Fisher Scientific) according to the manufacturer's instructions.

The composition of the isolated DNA and the proportion of linearized pGFP1 was analyzed by agarose gel electrophoresis and capillary electrophoresis. For the agarose gel electrophoresis, the isolated DNA was separated across a 1.5% agarose gel and then visualized using Midori Green (Nippon Genetics Europe) and a gel documentation system (BioRad).

The usability of the linearized DNA as template for the production of mRNA was verified by in vitro transcription as described in Example 2. The RNAs obtained were analyzed by polyacrylamide gel electrophoresis and the poly A tails of the mRNAs were analyzed by the described method with T4 RNA ligase 1 and RT-PCR.

### Example 5: Incorporation of the Cas9-CRISPR-tracrRNA Construct on the Chromosome of a Selected E. coli Strain

The CRISPR-Cas construct pCas9_tracrRNA_CRISPR (SEQ ID NO:43) described in Example 4 was integrated into the chromosome of E. coli NEB10Beta. The region chosen for the integration was an intergenic region of the gene locus of atpl. The Cas9-gRNA construct was integrated by using the method of gene deletion by λ Red recombination from Datsenko and Wanner (Datsenko et al. 2000, Proc. Natl. Acad. Sci. USA. 97:6640-5).

To integrate the Cas9-CRISPR-tracrRNA construct into the intergenic region of the atpl locus in the genome of the E. coli NEB10Beta strain used, the construct to be integrated was first designed. It contains not only the cas9_tracrRNA_CRISPR construct (SEQ ID NO: 43) described in Example 3, but also a chloramphenicol resistance cassette (cat), two FRT sites flanking the cat cassette, and flanking regions homologous to the integration site. The construct (SEQ ID NO:44) for genomic integration by λ Red recombination is schematically illustrated in FIG. 6. Two regions which are each 150 bp in length and homologous to the integration site flank the construct at the 3′ and 5′ ends. Present in between in the 5′-3′ orientation are the cas9_tracrRNA_CRISPR construct, a FRT site, the cat cassette and another FRT site.

The cat cassette serves for later selection to identify successful integration. The FRT sites are used later on to take the cat cassette out of the genome again with the aid of a recombinase. The homologous regions flanking the entire construct are 150 base pairs long and serve for integration at the target site. The construct is incorporated into the genome by homologous recombination.

The complete construct was synthesized by GeneArt and cloned into the vector pMX vector (GeneArt). From this plasmid, the construct was cut out using restriction enzymes (XhoI) and purified using the GeneJet Gel Extraction Kit (Thermo Fisher Scientific) according to the manufacturer's instructions.

Homologous recombination for integration was brought about by using the Lambda Red system, which is coded on the plasmid pKD46 (CGSC: 7736). The plasmid was transformed into NEB10Beta cells and competent cells are then produced as described by Datsenko and Wanner ((Datsenko et al. 2000, Proc. Natl. Acad. Sci. USA. 97:6640-5). Said competent cells were transformed with the purified integration construct. The selection for integration of the chloramphenicol resistance cassette (cat=chloramphenicol acetyltransferase) and thus also the Cas9 and guide RNA construct into the chromosome was done on LB agar plates containing 20 mg/l chloramphenicol. Integration at the desired position in the chromosome was checked/confirmed by PCR using the oligonucleotides Check Fwd (SEQ ID NO:54) and Check Rev (SEQ ID NO:55) and chromosomal DNA of the chloramphenicol-resistant cells as template. As negative control, PCR was also carried out with chromosomal DNA of the parent strain E. coli NEB10Beta. In this way, what were obtained were E. coli cells in which the Cas9 and guide RNA construct has been integrated into the genome.

Subsequently, the plasmid pKD46 was removed from the cells according to the procedure described by Datsenko and Wanner (see above) and the strain generated in this way was referred to as E. coli NEB10Beta atpl::cas9-CRISPR-tracrRNA-cat. The chloramphenicol resistance cassette was removed from the chromosome as specified by Datsenko and Wanner (see above) with the aid of the plasmid pCP20 (CGSC: 7629) containing the code of the FLP recombinase gene. The chloramphenicol-sensitive strain containing Cas9-CRISPR-tracrRNA that was ultimately obtained by this method was referred to as E. coli NEB10Beta atpl::cas9-CRISPR-tracrRNA. The successful integration of the construct (SEQ ID NO:44) was lastly verified by sequencing using different primers (SEQ ID NO:45-54).

The plasmid pGFP1 (SEQ ID NO:11) was then transformed into the new strain (E. coli NEB10Beta atpl::cas9-CRISPR-tracrRNA) in order to test the system. Following regeneration and selection (overnight on LB-ampicillin agar plates), a colony was selected and it was used to first prepare an overnight culture in 20 mL of LB-Amp (37 °C, 200 rpm). This preculture was used to inoculate the 100 mL main culture. The resultant main culture was incubated up to an OD₆₀₀ₙₘ of 0.8. Once the target OD was reached, IPTG was added to start the expression of both Cas9 and the guide RNA. After 2 h under inducing conditions, the culture was harvested by centrifugation. The plasmid DNA in the cells was subsequently isolated and purified using the Gene Jet Plasmid Kit (Thermo Fisher Scientific) according to the manufacturer's instructions.

The isolated plasmid DNA was analyzed by agarose gel electrophoresis and capillary electrophoresis. For the gel electrophoresis, the isolated plasmid DNA was separated according to size and property (linearized, coiled) by means of a 1.5% agarose gel and then visualized using Midori Green (Nippon Genetics Europe) on a gel documentation system (BioRad).

The usability of the DNA for the production of mRNA was checked by in vitro transcription as described in Example 2.

1-15. (canceled)

16. A method for producing a linear double-stranded DNA molecule, wherein the linear DNA molecule having blunt ends, and wherein the linear DNA molecule having a poly T sequence at the 5′ end of the template strand, wherein the process comprises the steps of:
(1) providing a double-stranded circular DNA molecule comprising on the template strand in the 3′ to 5′ direction:
(i) a template sequence operatively linked to an RNA polymerase promoter,
(ii) a poly T sequence,
(iii) a nucleotide sequence described by N₁N₂N₃, where N₁ to N₃ are each independently G, T, A or C, and
(iv) a protospacer adjacent motif (PAM);
(2) providing a guide RNA comprising a region which is complementary to the 5′ end of the poly T sequence (ii) and to the nucleotide sequence (iii) and a region which interacts with a Cas nuclease;
(3) providing a type II Cas nuclease; and
(4) contacting the double-stranded circular DNA molecule with the guide RNA and the CAS nuclease.

17. The method of claim 16, wherein the poly T sequence (ii) consists of between about 40 and about 250 T nucleotides, preferably between about 100 and about 140 T nucleotides, and particularly preferably about 120 T nucleotides.

18. The method of claim 16, wherein the region of the guide RNA which is complementary to the 5′ end of the poly T sequence (ii) and to the nucleotide sequence (iii) has the sequence 5′-G (A)ₙN₄N₅N₆-3′;
where n is a number between 17 and 23;
where N₄ to N₆ are each independently G, U, A or C; and
where the nucleotides N₄N₅N₆ are complementary to the nucleotides N₁N₂N₃ from (iii).

19. The method of claim 16, wherein the type II Cas nuclease is Cas9, preferably selected from Cas9 from Streptococcus pyogenes having SEQ ID NO:2 or a sequence at least 70% homologous thereto, Cas9 from Streptococcus aureus having SEQ ID NO:3 or a sequence at least 70% homologous thereto and Cas9 from Streptococcus thermophilus having SEQ ID NO:4 or a sequence at least 70% homologous thereto.

20. The method of claim 16, wherein the type II Cas nuclease is Cas9 from Streptococcus pyogenes having SEQ ID NO:2 or a sequence at least 70% homologous thereto.

21. The method of claim 16, wherein the PAM has the sequence 5′-NGG-3′; and
where N is independently selected from G, C, A and T.

22. The method of claim 16, wherein the poly T sequence (ii) consists of about 120 T nucleotides;
wherein the region of the guide RNA which is complementary to the 5′ end of the poly T sequence (ii) and to the nucleotide sequence (iii) has the sequence 5′-G (A) n-N₄N₅N₆-3′, where n is equal to 17, where N₄ to N₆ are each independently G, U or C;
wherein the PAM has the sequence 5′-NGG-3′, where N is independently selected from G, C, A and T; and
wherein the type II Cas nuclease is Cas9 from Streptococcus pyogenes having SEQ ID NO: 2 or a sequence at least 70% homologous thereto.

23. The method of claim 16, wherein the providing of the guide RNA step is achieved by the expression of a nucleotide sequence encoding the guide RNA.

24. The method of claim 23, wherein the nucleotide sequence encoding the guide RNA and/or the nucleotide sequence encoding the Cas nuclease is/are operatively linked to an inducible promoter.

25. The method of claim 25, wherein the inducible promoter is a lactose-inducible promoter, in particular the promoter WAC003 having the sequence SEQ ID NO:6 or the promoter WAC003-LacDel having the sequence SEQ ID NO:7.

26. The method of claim 16, wherein the providing of the type II Cas nuclease step is achieved by the expression of a nucleotide sequence encoding the Cas nuclease.

27. The method of claim 26, wherein the nucleotide sequence encoding the guide RNA and/or the nucleotide sequence encoding the Cas nuclease is/are operatively linked to an inducible promoter.

28. The method of claim 27, wherein the inducible promoter is a lactose-inducible promoter, in particular the promoter WAC003 having the sequence SEQ ID NO:6 or the promoter WAC003-LacDel having the sequence SEQ ID NO:7.

29. The method of claim 23, characterized in that the nucleotide sequence encoding the guide RNA and/or the nucleotide sequence encoding the Cas nuclease has/have been integrated into the genome of E. coli.

30. The method of claim 26, characterized in that the nucleotide sequence encoding the guide RNA and/or the nucleotide sequence encoding the Cas nuclease has/have been integrated into the genome of E. coli.

31. The method of claim 24, characterized in that the nucleotide sequence encoding the guide RNA and/or the nucleotide sequence encoding the Cas nuclease has/have been integrated into the genome of E. coli.

32. The method of claim 25, characterized in that the nucleotide sequence encoding the guide RNA and/or the nucleotide sequence encoding the Cas nuclease has/have been integrated into the genome of E. coli.

33. The method of claim 27, characterized in that the nucleotide sequence encoding the guide RNA and/or the nucleotide sequence encoding the Cas nuclease has/have been integrated into the genome of E. coli.

34. The method of claim 28, characterized in that the nucleotide sequence encoding the guide RNA and/or the nucleotide sequence encoding the Cas nuclease has/have been integrated into the genome of E. coli.

35. A use of a type II Cas nuclease for linearization of a double-stranded, circular DNA molecule, the linear DNA molecule comprising on the template strand in the 3′ to 5′ direction:
(i) a template sequence operatively linked to an RNA polymerase promoter,
(ii) a poly T sequence,
(iii) a nucleotide sequence described by N₁N₂N₃, where N₁ to N₃ are each independently G, T, A or C, and
(iv) a protospacer adjacent motif (PAM).

36. A use of a recombinant microorganism in which the nucleotide sequence encoding a guide RNA and/or the nucleotide sequence encoding a type II Cas nuclease has/have been integrated into the genome, for production of a linear double-stranded DNA molecule, the linear DNA molecule having blunt ends, and the linear DNA molecule having a poly T sequence at the 5′ end of the template strand.

37. The use of claim 36, wherein the recombinant microorganism is E. coli, preferably an E. coli K12 strain, particularly preferably E. coli NEB10Beta.


SEQUENCE LISTING (55 sequences)

SEQ ID NO: 1 - RNA, 101 nt, synthetic construct
  source 1..101: mol_type = other RNA
gaaaaaaaaaaaaaaaaagtcgttttagagctagaaatagcaagttaaaataaggctagtccgttatcaacttgaaaaagtggcaccgagtcggtgctttt

SEQ ID NO: 2 - protein, 1368 aa, Streptococcus pyogenes [sequence omitted]

SEQ ID NO: 3 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 4 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 5 - DNA, 5545 bp, synthetic construct
  source 1..5545: mol_type = other DNA
ttgacaattaatcatcggctcgtataatgtgtggaattgtgagcggataacaatttcacacaggaaacagaattctaaggaggaaattatatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatctagaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcggccgcatatgagctctccggaagatctctcgaggtcgacgtacgtgcccatggatgcatttaattaattcgaagttaacttaagcttgtacactagtaaagttctgctatgtggcgcggtattatcccgtgttgacgccgggcaagagcaactcggtcgccgcatacactattctcagaatgacttggttgagtccatggcggccgccttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagctagatttcagtgcaatttatctcttcaaatgtagcacctgaagtcagccccatacgatataagttgtaattctcatgtttgacagcttatcatcgataagctccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatggcctgtcgcttgcggtattcggaatcttgcacgccctcgctcaagccttcgtcactggtcccgccaccaaacgtttcggcgagaagcaggccattatcgccggcatggcggccgacgcgctgggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattgtggaattgtgagcgg

SEQ ID NO: 6 - DNA, 1452 bp, synthetic construct
  source 1..1452: mol_type = other DNA
tgaaattgttatccgctcacaattccacacattatacgagccgatgattaattgtcaaccgctcacaattccacaatatttgccagaaccgttatgatgtcggcgcaaaaaacattatccagaacgggagtgcgccttgagcgacacgaattatgcagtgatttacgacctgcacagccataccacagcttccgatggctgcctgacgccagaagcattggtgcaccgtgcagtcgataagcccggatcagcttgcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcag

SEQ ID NO: 7 - DNA, 1386 bp, synthetic construct
  source 1..1386: mol_type = other DNA
agaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattgtggaattgtgagcggttgacaattaatcatcggctcgtataatgtgtggaattgtgagcggataacaatttca

SEQ ID NO: 8 - DNA, 67 bp, synthetic construct
  source 1..67: mol_type = other DNA
gaatccgaattctaatacgactcactatagggataaaggaggtaaataatggctagcaaaggagaag

SEQ ID NO: 9 - DNA, 100 bp, synthetic construct
  source 1..100: mol_type = other DNA
ttgcactctagagccgacttttttttttttttttttttttttttttttttttttttttttttttttttttttttttttttatttgtagagctcatccatg

SEQ ID NO: 10 - DNA, 846 bp, synthetic construct
  source 1..846: mol_type = other DNA
gaatccgaattctaatacgactcactatagggataaaggaggtaaataatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataaaaaaaaaaaaaaaaaaaaaaaaaaaaaaaaaaaaaaaaaaaaaaaaaaaaaaaaaaaaaagtcggctctagagtgcaa

SEQ ID NO: 11 - DNA, 3487 bp, synthetic construct
  source 1..3487: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccgaattctaatacgactcactatagggataaaggaggtaaataatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataaaaaaaaaaaaaaaaaaaaaaaaaaaaaaaaaaaaaaaaaaaaaaaaaaaaaaaaaaaaaagtcggctctagagtcgacctgcaggcatgcaagcttactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 12 - DNA, 121 bp, synthetic construct
  source 1..121: mol_type = other DNA
aaaagcaccgactcggtgccactttttcaagttgataacggactagccttattttaacttgctatttctagctctaaaacgactttttttttttttttttctatagtgagtcgtattagaa

SEQ ID NO: 13 - DNA, 21 bp, synthetic construct
  source 1..21: mol_type = other DNA
ttctaatacgactcactatag

SEQ ID NO: 14 - DNA, 3489 bp, synthetic construct
  source 1..3489: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccgaattctaatacgactcactatagggataaaggaggtaaataatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataaaaaaaaaaaaaaaaaaaaaaaaaaaaaaaaaaaaaaaaaaaaaaaaaaaaaaaaaaaaaaaagtcttctctagagtcgacctgcaggcatgcaagcttactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 15 - DNA, 102 bp, synthetic construct
  source 1..102: mol_type = other DNA
ttgcactctagagaagacttttttttttttttttttttttttttttttttttttttttttttttttttttttttttttttttatttgtagagctcatccatg

SEQ ID NO: 16 - DNA, 828 bp, synthetic construct
  source 1..828: mol_type = other DNA
gaatccgaattctaatacgactcactatagggataaaggaggtaaataatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataaaaaaaaaaaaaaaaaaaaaaaaaaaaaaaaaaaaaaaaaaaaaaaaaaaaaaaaaaaaaa

SEQ ID NO: 17 - DNA, 67 bp, synthetic construct; identical to SEQ ID NO: 8
  source 1..67: mol_type = other DNA

SEQ ID NO: 18 - DNA, 82 bp, synthetic construct
  source 1..82: mol_type = other DNA
ttttttttttttttttttttttttttttttttttttttttttttttttttttttttttttttatttgtagagctcatccatg

SEQ ID NO: 19 - DNA, 18 bp, synthetic construct
  source 1..18: mol_type = other DNA
tgtgcggcattcttatta

SEQ ID NO: 20 - DNA, 21 bp, synthetic construct
  source 1..21: mol_type = other DNA
catggcatggatgagctctac

SEQ ID NO: 21 - DNA, 18 bp, synthetic construct
  source 1..18: mol_type = other DNA
taataagaatgccgcaca

SEQ ID NO: 22 - DNA, 46 bp, synthetic construct
  source 1..46: mol_type = other DNA
atccacgaattcaggaggcaaaaatggacaaaaaatacagcattgg

SEQ ID NO: 23 - DNA, 129 bp, synthetic construct
  source 1..129: mol_type = other DNA
atccactctagaatccacggtaccatccacatccacatccacatccacacagggataaaaaaagcagcactgtaatcagtgctgctttgtcgtttttttcatcatcatcaatcaccacctaactggctc

SEQ ID NO: 24 - DNA, 10884 bp, synthetic construct
  source 1..10884: mol_type = other DNA
catggggtttgcagtcagagtagaatagaagtatcaaaaaaagcaccgactcggtgccactttttcaagttgataacggactagccttattttaacttgctatgctgttttgaatggttccaacaagattattttataacttttataacaaataatcaaggagaaattcaaagaaatttatcagccataaaacaatacttaatactatagaatgataacaaaataaactactttttaaaagaattttgtgttataatctatttattattaagtattgggtaatattttttgaagagatattttgaaaaagaaaaattaaagcatattaaactaatttcggaggtcattaaaactattattgaaatcatcaaactcattatggatttaatttaaactttttattttaggaggcaaaaatggacaaaaaatacagcattggtctggatattggcaccaatagcgttggttgggcagttattaccgatgaatataaagttccgagcaaaaaattcaaggtgctgggtaataccgatcgccatagcattaaaaagaatctgattggtgcactgctgttcgatagcggtgaaaccgcagaagcaacccgtctgaaacgtaccgcacgtcgtcgttatacccgtcgtaaaaatcgtatttgctatctgcaagagatctttagcaacgaaatggccaaagtggatgatagcttttttcatcgtctggaagaatcctttctggtggaagaagataaaaaacacgaacgccatccgatttttggcaacattgttgatgaagtggcctaccatgaaaaatatccgaccatttatcatctgcgcaaaaaactggttgatagcaccgataaagccgatctgcgtctgatttatctggcactggcacatatgatcaaatttcgtggtcattttctgatcgaaggtgatctgaatccggataatagtgatgtggataaactgttcattcagctggtgcagacctataatcagctgtttgaagaaaatccgattaatgccagcggtgttgatgcaaaagcaattctgagcgcacgtctgagcaaaagccgtcgcctggaaaacctgattgcacagctgcctggtgaaaaaaagaatggtctgtttggtaatctgatcgcactgagcctgggtctgaccccgaactttaaatcaaattttgatctggccgaagatgccaaactgcagctgagcaaagatacctatgatgatgatctggataatctgctggcacagattggtgatcagtatgcagacctgtttctggcagcgaaaaatctgagtgatgcaattctgctgagcgatattctgcgtgttaataccgaaattaccaaagcaccgctgagcgcaagtatgattaaacgttatgatgaacatcaccaggatctgaccctgctgaaagcactggttcgtcagcagctgccggaaaagtataaagaaatctttttcgaccagagcaagaatggctatgccggttatattgatggtggtgcaagccaagaagagttctacaaattcattaaaccgatcctggaaaagatggatggcaccgaagaactgctggttaaactgaatcgtgaagatctgctgcgtaaacagcgtacctttgataatggtagcattccgcatcagattcatctgggtgaactgcatgccattctgcgtcgtcaagaagatttttatccgttcctgaaagataaccgcgagaaaatcgaaaaaattctgacctttcgcatcccgtattatgtgggtccgctggcacgtggtaatagccgttttgcatggatgacccgtaaaagcgaagaaaccattacaccgtggaatttcgaagaggttgttgataaaggtgcaagcgcacagagctttattgaacgtatgaccaacttcgataaaaacctgccgaatgaaaaagttctgccgaaacatagcctgctgtatgaatatttcaccgtgtataatgagctgaccaaagtgaaatatgtgaccgaaggtatgcgtaaaccggcatttctgagcggtgaacagaaaaaagcgattgttgatctgctgtttaaaaccaatcgtaaggttaccgttaagcagctgaaagaggattatttcaaaaaaatcgagtgctttgatagcgtcgaaattagcggtgtggaagatcgttttaatgcaagcctgggcacctatcacgatctgctgaaaatcattaaagataaagattttctggacaacgaagagaacgaggacattctggaagatattgttctgacactgaccctgtttgaggatcgtgaaatgattgaagaacgcctgaaaacatatgcccacctgttcgatgataaagtgatgaaacaactgaaacgccgtcgctataccggttggggtcgtctgagccgtaaactgattaatggtattcgtgataaacagagcggcaaaaccattctggattttctgaaaagtgatggctttgccaatcgcaattttatgcagctgattcatgatgatagcctgacctttaaagaggatattcagaaagcacaggttagcggtcagggtgatagtctgcatgaacatattgcaaatctggcaggtagtccggcaatcaaaaaaggtattctgcagaccgttaaagtggttgatgaactggttaaagttatgggtcgtcataaaccggaaaacatcgtgattgaaatggcacgtgaaaatcagaccacacagaaaggccagaaaaatagccgtgaacgcatgaaacgtattgaagaaggtattaaagaactgggcagccagattctgaaagaacatccggttgaaaatacccagctgcagaacgaaaaactgtatctgtattacctgcagaatggtcgtgacatgtatgttgatcaagaactggatattaatcgcctgagcgattatgatgtcgatcatattgttccgcagtcctttcttaaagatgatagcattgacaacaaagtgctgacccgtagcgacaaaaatcgtggtaaaagtgataatgtgccgagcgaagaggtggttaaaaagatgaaaaactattggcgtcaactgctgaacgccaaactgatcacacagcgcaaatttgataatctgacaaaagcagaacgtggtggtctgagcgagctggataaagcaggttttatcaaacgtcagctggttgaaacccgtcagatcacaaaacatgttgcacagattctggatagccgcatgaataccaaatatgacgaaaacgataaactgatccgcgaggttaaagtgattaccctgaaaagcaaactggtgagcgattttcgtaaagacttccagttttacaaagtgcgcgaaatcaacaattatcatcacgcacatgatgcctatctgaatgcagttgttggcaccgcactgattaagaaatatcctaaactggaaagcgagttcgtgtatggcgattataaagtttatgatgtgcggaagatgatcgccaaaagcgaacaagaaattggtaaagcaaccgccaaatacttcttctacagcaatattatgaacttcttcaaaaccgaaatcaccctggccaatggtgaaattcgtaaacgtccgctgatcgaaaccaatggcgaaaccggtgaaattgtttgggataaaggtcgtgattttgccaccgttcgtaaagttctgagtatgccgcaggttaacattgtgaaaaagaccgaagttcagaccggtggttttagcaaagaaagcattctgcctaaacgcaatagcgataagctgattgcccgtaaaaaagattgggacccgaaaaaatacggtggatttgatagcccgaccgttgcatatagcgttctggttgttgcaaaagtggaaaaaggtaagtccaagaaactgaaaagcgttaaagagctgctgggcattaccattatggaacgtagcagctttgaaaaaaacccgatcgatttcctggaagccaaaggttataaagaggtgaagaaagacctgattatcaagctgccaaagtatagcctgtttgaactggaaaatggtcgtaaacgcatgctggcaagtgcaggcgaactgcagaaaggtaatgaactggcactgccgagcaaatatgttaactttctgtatctggccagccactatgaaaagctgaaaggttcaccggaagataacgaacagaaacagttatttgtggaacagcacaaacactatctggatgaaattattgagcagatcagcgaatttagcaaacgtgttattctggcagatgccaatctggacaaagttctgtcagcatataacaaacaccgcgataaaccgattcgtgaacaggcagaaaacatcattcacctgtttaccctgaccaatctgggtgcaccggcagcctttaaatactttgataccaccattgatcgcaaacgttataccagcaccaaagaagttctggacgcaaccctgattcatcagagcattaccggtctgtatgaaacccgcattgatctgagccagttaggtggtgattgaagtatattttagatgaagattatttcttaatccatgggtatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgcatcgatttattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattcgagctctaaggaggttataaaaaatggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataaaacgaatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatgacgcatcctcacgataatatccgggtaggcgcaatcactttcgtctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaatc

SEQ ID NO: 25 - DNA, 9023 bp, synthetic construct
  source 1..9023: mol_type = other DNA
ttgacaattaatcatcggctcgtataatgtgtggaattgtgagcggataacaatttcacacaggaaacagaattcaggaggcaaaaatggacaaaaaatacagcattggtctggatattggcaccaatagcgttggttgggcagttattaccgatgaatataaagttccgagcaaaaaattcaaggtgctgggtaataccgatcgccatagcattaaaaagaatctgattggtgcactgctgttcgatagcggtgaaaccgcagaagcaacccgtctgaaacgtaccgcacgtcgtcgttatacccgtcgtaaaaatcgtatttgctatctgcaagagatctttagcaacgaaatggccaaagtggatgatagcttttttcatcgtctggaagaatcctttctggtggaagaagataaaaaacacgaacgccatccgatttttggcaacattgttgatgaagtggcctaccatgaaaaatatccgaccatttatcatctgcgcaaaaaactggttgatagcaccgataaagccgatctgcgtctgatttatctggcactggcacatatgatcaaatttcgtggtcattttctgatcgaaggtgatctgaatccggataatagtgatgtggataaactgttcattcagctggtgcagacctataatcagctgtttgaagaaaatccgattaatgccagcggtgttgatgcaaaagcaattctgagcgcacgtctgagcaaaagccgtcgcctggaaaacctgattgcacagctgcctggtgaaaaaaagaatggtctgtttggtaatctgatcgcactgagcctgggtctgaccccgaactttaaatcaaattttgatctggccgaagatgccaaactgcagctgagcaaagatacctatgatgatgatctggataatctgctggcacagattggtgatcagtatgcagacctgtttctggcagcgaaaaatctgagtgatgcaattctgctgagcgatattctgcgtgttaataccgaaattaccaaagcaccgctgagcgcaagtatgattaaacgttatgatgaacatcaccaggatctgaccctgctgaaagcactggttcgtcagcagctgccggaaaagtataaagaaatctttttcgaccagagcaagaatggctatgccggttatattgatggtggtgcaagccaagaagagttctacaaattcattaaaccgatcctggaaaagatggatggcaccgaagaactgctggttaaactgaatcgtgaagatctgctgcgtaaacagcgtacctttgataatggtagcattccgcatcagattcatctgggtgaactgcatgccattctgcgtcgtcaagaagatttttatccgttcctgaaagataaccgcgagaaaatcgaaaaaattctgacctttcgcatcccgtattatgtgggtccgctggcacgtggtaatagccgttttgcatggatgacccgtaaaagcgaagaaaccattacaccgtggaatttcgaagaggttgttgataaaggtgcaagcgcacagagctttattgaacgtatgaccaacttcgataaaaacctgccgaatgaaaaagttctgccgaaacatagcctgctgtatgaatatttcaccgtgtataatgagctgaccaaagtgaaatatgtgaccgaaggtatgcgtaaaccggcatttctgagcggtgaacagaaaaaagcgattgttgatctgctgtttaaaaccaatcgtaaggttaccgttaagcagctgaaagaggattatttcaaaaaaatcgagtgctttgatagcgtcgaaattagcggtgtggaagatcgttttaatgcaagcctgggcacctatcacgatctgctgaaaatcattaaagataaagattttctggacaacgaagagaacgaggacattctggaagatattgttctgacactgaccctgtttgaggatcgtgaaatgattgaagaacgcctgaaaacatatgcccacctgttcgatgataaagtgatgaaacaactgaaacgccgtcgctataccggttggggtcgtctgagccgtaaactgattaatggtattcgtgataaacagagcggcaaaaccattctggattttctgaaaagtgatggctttgccaatcgcaattttatgcagctgattcatgatgatagcctgacctttaaagaggatattcagaaagcacaggttagcggtcagggtgatagtctgcatgaacatattgcaaatctggcaggtagtccggcaatcaaaaaaggtattctgcagaccgttaaagtggttgatgaactggttaaagttatgggtcgtcataaaccggaaaacatcgtgattgaaatggcacgtgaaaatcagaccacacagaaaggccagaaaaatagccgtgaacgcatgaaacgtattgaagaaggtattaaagaactgggcagccagattctgaaagaacatccggttgaaaatacccagctgcagaacgaaaaactgtatctgtattacctgcagaatggtcgtgacatgtatgttgatcaagaactggatattaatcgcctgagcgattatgatgtcgatcatattgttccgcagtcctttcttaaagatgatagcattgacaacaaagtgctgacccgtagcgacaaaaatcgtggtaaaagtgataatgtgccgagcgaagaggtggttaaaaagatgaaaaactattggcgtcaactgctgaacgccaaactgatcacacagcgcaaatttgataatctgacaaaagcagaacgtggtggtctgagcgagctggataaagcaggttttatcaaacgtcagctggttgaaacccgtcagatcacaaaacatgttgcacagattctggatagccgcatgaataccaaatatgacgaaaacgataaactgatccgcgaggttaaagtgattaccctgaaaagcaaactggtgagcgattttcgtaaagacttccagttttacaaagtgcgcgaaatcaacaattatcatcacgcacatgatgcctatctgaatgcagttgttggcaccgcactgattaagaaatatcctaaactggaaagcgagttcgtgtatggcgattataaagtttatgatgtgcggaagatgatcgccaaaagcgaacaagaaattggtaaagcaaccgccaaatacttcttctacagcaatattatgaacttcttcaaaaccgaaatcaccctggccaatggtgaaattcgtaaacgtccgctgatcgaaaccaatggcgaaaccggtgaaattgtttgggataaaggtcgtgattttgccaccgttcgtaaagttctgagtatgccgcaggttaacattgtgaaaaagaccgaagttcagaccggtggttttagcaaagaaagcattctgcctaaacgcaatagcgataagctgattgcccgtaaaaaagattgggacccgaaaaaatacggtggatttgatagcccgaccgttgcatatagcgttctggttgttgcaaaagtggaaaaaggtaagtccaagaaactgaaaagcgttaaagagctgctgggcattaccattatggaacgtagcagctttgaaaaaaacccgatcgatttcctggaagccaaaggttataaagaggtgaagaaagacctgattatcaagctgccaaagtatagcctgtttgaactggaaaatggtcgtaaacgcatgctggcaagtgcaggcgaactgcagaaaggtaatgaactggcactgccgagcaaatatgttaactttctgtatctggccagccactatgaaaagctgaaaggttcaccggaagataacgaacagaaacagttatttgtggaacagcacaaacactatctggatgaaattattgagcagatcagcgaatttagcaaacgtgttattctggcagatgccaatctggacaaagttctgtcagcatataacaaacaccgcgataaaccgattcgtgaacaggcagaaaacatcattcacctgtttaccctgaccaatctgggtgcaccggcagcctttaaatactttgataccaccattgatcgcaaacgttataccagcaccaaagaagttctggacgcaaccctgattcatcagagcattaccggtctgtatgaaacccgcattgatctgagccagttaggtggtgattgatgatgatgaaaaaaacgacaaagcagcactgattacagtgctgctttttttatccctgtgtggatgtggatgtggatgtggatggtaccgtggattctagaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcggccgcatatgagctctccggaagatctctcgaggtcgacgtacgtgcccatggatgcatttaattaattcgaagttaacttaagcttgtacactagtaaagttctgctatgtggcgcggtattatcccgtgttgacgccgggcaagagcaactcggtcgccgcatacactattctcagaatgacttggttgagtccatggcggccgccttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagctagatttcagtgcaatttatctcttcaaatgtagcacctgaagtcagccccatacgatataagttgtaattctcatgtttgacagcttatcatcgataagctccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatggcctgtcgcttgcggtattcggaatcttgcacgccctcgctcaagccttcgtcactggtcccgccaccaaacgtttcggcgagaagcaggccattatcgccggcatggcggccgacgcgctgggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattgtggaattgtgagcgg

SEQ ID NO: 26 - DNA, 18 bp, synthetic construct
  source 1..18: mol_type = other DNA
catcataacggttctggc

SEQ ID NO: 27 - DNA, 20 bp, synthetic construct
  source 1..20: mol_type = other DNA
ctgcgttctgatttaatctg

SEQ ID NO: 28 - DNA, 196 bp, synthetic construct
  source 1..196: mol_type = other DNA
ttgacaattaatcatcggctcgtataatgtgtggaattgtgagcggataacaatttgttggaaccattcaaaacagcatagcaagttaaaataaggctagtccgttatcaacttgaaaaagtggcaccgagtcggtgcttttttgaaaaaaacgacaaagcagcactgattacagtgctgctttttttatccctgt

SEQ ID NO: 29 - DNA, 211 bp, synthetic construct
  source 1..211: mol_type = other DNA
ttgacaattaatcatcggctcgtataatgtgtggaattgtgagcggataacaatttgttttagagctatgctgttttgaatggtcccaaaacaaaaaaaaaaaaaaaaaaaaaaaaaaagtcgttttagagctatgctgttttgaatggtcccaaaactgaaaaaaacgacaaagcagcactgattacagtgctgctttttttatccctgt

SEQ ID NO: 30 - DNA, 32 bp, synthetic construct
  source 1..32: mol_type = other DNA
ttgcacggtaccttgacaattaatcatcggct

SEQ ID NO: 31 - DNA, 56 bp, synthetic construct
  source 1..56: mol_type = other DNA
aaattgttatccgctcacaattccacacattatacgagccgatgattaattgtcaa

SEQ ID NO: 32 - DNA, 60 bp, synthetic construct
  source 1..60: mol_type = other DNA
ttgtgagcggataacaatttgttggaaccattcaaaacagcatagcaagttaaaataagg

SEQ ID NO: 33 - DNA, 60 bp, synthetic construct
  source 1..60: mol_type = other DNA
accgactcggtgccactttttcaagttgataacggactagccttattttaacttgctatg

SEQ ID NO: 34 - DNA, 60 bp, synthetic construct
  source 1..60: mol_type = other DNA
aaaagtggcaccgagtcggtgcttttttgaaaaaaacgacaaagcagcactgattacagt

SEQ ID NO: 35 - DNA, 88 bp, synthetic construct
  source 1..88: mol_type = other DNA
ttgcactctagaatccaccccgggatccacatccacatccacatccacacagggataaaaaaagcagcactgtaatcagtgctgcttt

SEQ ID NO: 36 - DNA, 32 bp, synthetic construct
  source 1..32: mol_type = other DNA
ttgcaccccgggttgacaattaatcatcggct

SEQ ID NO: 37 - DNA, 60 bp, synthetic construct
  source 1..60: mol_type = other DNA
aaacaaattgttatccgctcacaattccacacattatacgagccgatgattaattgtcaa

SEQ ID NO: 38 - DNA, 60 bp, synthetic construct
  source 1..60: mol_type = other DNA
gagcggataacaatttgttttagagctatgctgttttgaatggtcccaaaacaaaaaaaa

SEQ ID NO: 39 - DNA, 60 bp, synthetic construct
  source 1..60: mol_type = other DNA
acagcatagctctaaaacgactttttttttttttttttttttttttttgttttgggacca

SEQ ID NO: 40 - DNA, 60 bp, synthetic construct
  source 1..60: mol_type = other DNA
tcgttttagagctatgctgttttgaatggtcccaaaactgaaaaaaacgacaaagcagca

SEQ ID NO: 41 - DNA, 63 bp, synthetic construct
  source 1..63: mol_type = other DNA
ttgcactctagaacagggataaaaaaagcagcactgtaatcagtgctgctttgtcgttttttt

SEQ ID NO: 42 - DNA, 9249 bp, synthetic construct
  source 1..9249: mol_type = other DNA
ttgacaattaatcatcggctcgtataatgtgtggaattgtgagcggataacaatttcacacaggaaacagaattcaggaggcaaaaatggacaaaaaatacagcattggtctggatattggcaccaatagcgttggttgggcagttattaccgatgaatataaagttccgagcaaaaaattcaaggtgctgggtaataccgatcgccatagcattaaaaagaatctgattggtgcactgctgttcgatagcggtgaaaccgcagaagcaacccgtctgaaacgtaccgcacgtcgtcgttatacccgtcgtaaaaatcgtatttgctatctgcaagagatctttagcaacgaaatggccaaagtggatgatagcttttttcatcgtctggaagaatcctttctggtggaagaagataaaaaacacgaacgccatccgatttttggcaacattgttgatgaagtggcctaccatgaaaaatatccgaccatttatcatctgcgcaaaaaactggttgatagcaccgataaagccgatctgcgtctgatttatctggcactggcacatatgatcaaatttcgtggtcattttctgatcgaaggtgatctgaatccggataatagtgatgtggataaactgttcattcagctggtgcagacctataatcagctgtttgaagaaaatccgattaatgccagcggtgttgatgcaaaagcaattctgagcgcacgtctgagcaaaagccgtcgcctggaaaacctgattgcacagctgcctggtgaaaaaaagaatggtctgtttggtaatctgatcgcactgagcctgggtctgaccccgaactttaaatcaaattttgatctggccgaagatgccaaactgcagctgagcaaagatacctatgatgatgatctggataatctgctggcacagattggtgatcagtatgcagacctgtttctggcagcgaaaaatctgagtgatgcaattctgctgagcgatattctgcgtgttaataccgaaattaccaaagcaccgctgagcgcaagtatgattaaacgttatgatgaacatcaccaggatctgaccctgctgaaagcactggttcgtcagcagctgccggaaaagtataaagaaatctttttcgaccagagcaagaatggctatgccggttatattgatggtggtgcaagccaagaagagttctacaaattcattaaaccgatcctggaaaagatggatggcaccgaagaactgctggttaaactgaatcgtgaagatctgctgcgtaaacagcgtacctttgataatggtagcattccgcatcagattcatctgggtgaactgcatgccattctgcgtcgtcaagaagatttttatccgttcctgaaagataaccgcgagaaaatcgaaaaaattctgacctttcgcatcccgtattatgtgggtccgctggcacgtggtaatagccgttttgcatggatgacccgtaaaagcgaagaaaccattacaccgtggaatttcgaagaggttgttgataaaggtgcaagcgcacagagctttattgaacgtatgaccaacttcgataaaaacctgccgaatgaaaaagttctgccgaaacatagcctgctgtatgaatatttcaccgtgtataatgagctgaccaaagtgaaatatgtgaccgaaggtatgcgtaaaccggcatttctgagcggtgaacagaaaaaagcgattgttgatctgctgtttaaaaccaatcgtaaggttaccgttaagcagctgaaagaggattatttcaaaaaaatcgagtgctttgatagcgtcgaaattagcggtgtggaagatcgttttaatgcaagcctgggcacctatcacgatctgctgaaaatcattaaagataaagattttctggacaacgaagagaacgaggacattctggaagatattgttctgacactgaccctgtttgaggatcgtgaaatgattgaagaacgcctgaaaacatatgcccacctgttcgatgataaagtgatgaaacaactgaaacgccgtcgctataccggttggggtcgtctgagccgtaaactgattaatggtattcgtgataaacagagcggcaaaaccattctggattttctgaaaagtgatggctttgccaatcgcaattttatgcagctgattcatgatgatagcctgacctttaaagaggatattcagaaagcacaggttagcggtcagggtgatagtctgcatgaacatattgcaaatctggcaggtagtccggcaatcaaaaaaggtattctgcagaccgttaaagtggttgatgaactggttaaagttatgggtcgtcataaaccggaaaacatcgtgattgaaatggcacgtgaaaatcagaccacacagaaaggccagaaaaatagccgtgaacgcatgaaacgtattgaagaaggtattaaagaactgggcagccagattctgaaagaacatccggttgaaaatacccagctgcagaacgaaaaactgtatctgtattacctgcagaatggtcgtgacatgtatgttgatcaagaactggatattaatcgcctgagcgattatgatgtcgatcatattgttccgcagtcctttcttaaagatgatagcattgacaacaaagtgctgacccgtagcgacaaaaatcgtggtaaaagtgataatgtgccgagcgaagaggtggttaaaaagatgaaaaactattggcgtcaactgctgaacgccaaactgatcacacagcgcaaatttgataatctgacaaaagcagaacgtggtggtctgagcgagctggataaagcaggttttatcaaacgtcagctggttgaaacccgtcagatcacaaaacatgttgcacagattctggatagccgcatgaataccaaatatgacgaaaacgataaactgatccgcgaggttaaagtgattaccctgaaaagcaaactggtgagcgattttcgtaaagacttccagttttacaaagtgcgcgaaatcaacaattatcatcacgcacatgatgcctatctgaatgcagttgttggcaccgcactgattaagaaatatcctaaactggaaagcgagttcgtgtatggcgattataaagtttatgatgtgcggaagatgatcgccaaaagcgaacaagaaattggtaaagcaaccgccaaatacttcttctacagcaatattatgaacttcttcaaaaccgaaatcaccctggccaatggtgaaattcgtaaacgtccgctgatcgaaaccaatggcgaaaccggtgaaattgtttgggataaaggtcgtgattttgccaccgttcgtaaagttctgagtatgccgcaggttaacattgtgaaaaagaccgaagttcagaccggtggttttagcaaagaaagcattctgcctaaacgcaatagcgataagctgattgcccgtaaaaaagattgggacccgaaaaaatacggtggatttgatagcccgaccgttgcatatagcgttctggttgttgcaaaagtggaaaaaggtaagtccaagaaactgaaaagcgttaaagagctgctgggcattaccattatggaacgtagcagctttgaaaaaaacccgatcgatttcctggaagccaaaggttataaagaggtgaagaaagacctgattatcaagctgccaaagtatagcctgtttgaactggaaaatggtcgtaaacgcatgctggcaagtgcaggcgaactgcagaaaggtaatgaactggcactgccgagcaaatatgttaactttctgtatctggccagccactatgaaaagctgaaaggttcaccggaagataacgaacagaaacagttatttgtggaacagcacaaacactatctggatgaaattattgagcagatcagcgaatttagcaaacgtgttattctggcagatgccaatctggacaaagttctgtcagcatataacaaacaccgcgataaaccgattcgtgaacaggcagaaaacatcattcacctgtttaccctgaccaatctgggtgcaccggcagcctttaaatactttgataccaccattgatcgcaaacgttataccagcaccaaagaagttctggacgcaaccctgattcatcagagcattaccggtctgtatgaaacccgcattgatctgagccagttaggtggtgattgatgatgatgaaaaaaacgacaaagcagcactgattacagtgctgctttttttatccctgtgtggatgtggatgtggatgtggatggtaccttgacaattaatcatcggctcgtataatgtgtggaattgtgagcggataacaatttgttggaaccattcaaaacagcatagcaagttaaaataaggctagtccgttatcaacttgaaaaagtggcaccgagtcggtgcttttttgaaaaaaacgacaaagcagcactgattacagtgctgctttttttatccctgtgtggatgtggatgtggatgtggatcccggggtggattctagaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcggccgcatatgagctctccggaagatctctcgaggtcgacgtacgtgcccatggatgcatttaattaattcgaagttaacttaagcttgtacactagtaaagttctgctatgtggcgcggtattatcccgtgttgacgccgggcaagagcaactcggtcgccgcatacactattctcagaatgacttggttgagtccatggcggccgccttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagctagatttcagtgcaatttatctcttcaaatgtagcacctgaagtcagccccatacgatataagttgtaattctcatgtttgacagcttatcatcgataagctccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatggcctgtcgcttgcggtattcggaatcttgcacgccctcgctcaagccttcgtcactggtcccgccaccaaacgtttcggcgagaagcaggccattatcgccggcatggcggccgacgcgctgggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattgtggaattgtgagcgg

SEQ ID NO: 43 - DNA, 9454 bp, synthetic construct
  source 1..9454: mol_type = other DNA
ttgacaattaatcatcggctcgtataatgtgtggaattgtgagcggataacaatttcacacaggaaacagaattcaggaggcaaaaatggacaaaaaatacagcattggtctggatattggcaccaatagcgttggttgggcagttattaccgatgaatataaagttccgagcaaaaaattcaaggtgctgggtaataccgatcgccatagcattaaaaagaatctgattggtgcactgctgttcgatagcggtgaaaccgcagaagcaacccgtctgaaacgtaccgcacgtcgtcgttatacccgtcgtaaaaatcgtatttgctatctgcaagagatctttagcaacgaaatggccaaagtggatgatagcttttttcatcgtctggaagaatcctttctggtggaagaagataaaaaacacgaacgccatccgatttttggcaacattgttgatgaagtggcctaccatgaaaaatatccgaccatttatcatctgcgcaaaaaactggttgatagcaccgataaagccgatctgcgtctgatttatctggcactggcacatatgatcaaatttcgtggtcattttctgatcgaaggtgatctgaatccggataatagtgatgtggataaactgttcattcagctggtgcagacctataatcagctgtttgaagaaaatccgattaatgccagcggtgttgatgcaaaagcaattctgagcgcacgtctgagcaaaagccgtcgcctggaaaacctgattgcacagctgcctggtgaaaaaaagaatggtctgtttggtaatctgatcgcactgagcctgggtctgaccccgaactttaaatcaaattttgatctggccgaagatgccaaactgcagctgagcaaagatacctatgatgatgatctggataatctgctggcacagattggtgatcagtatgcagacctgtttctggcagcgaaaaatctgagtgatgcaattctgctgagcgatattctgcgtgttaataccgaaattaccaaagcaccgctgagcgcaagtatgattaaacgttatgatgaacatcaccaggatctgaccctgctgaaagcactggttcgtcagcagctgccggaaaagtataaagaaatctttttcgaccagagcaagaatggctatgccggttatattgatggtggtgcaagccaagaagagttctacaaattcattaaaccgatcctggaaaagatggatggcaccgaagaactgctggttaaactgaatcgtgaagatctgctgcgtaaacagcgtacctttgataatggtagcattccgcatcagattcatctgggtgaactgcatgccattctgcgtcgtcaagaagatttttatccgttcctgaaagataaccgcgagaaaatcgaaaaaattctgacctttcgcatcccgtattatgtgggtccgctggcacgtggtaatagccgttttgcatggatgacccgtaaaagcgaagaaaccattacaccgtggaatttcgaagaggttgttgataaaggtgcaagcgcacagagctttattgaacgtatgaccaacttcgataaaaacctgccgaatgaaaaagttctgccgaaacatagcctgctgtatgaatatttcaccgtgtataatgagctgaccaaagtgaaatatgtgaccgaaggtatgcgtaaaccggcatttctgagcggtgaacagaaaaaagcgattgttgatctgctgtttaaaaccaatcgtaaggttaccgttaagcagctgaaagaggattatttcaaaaaaatcgagtgctttgatagcgtcgaaattagcggtgtggaagatcgttttaatgcaagcctgggcacctatcacgatctgctgaaaatcattaaagataaagattttctggacaacgaagagaacgaggacattctggaagatattgttctgacactgaccctgtttgaggatcgtgaaatgattgaagaacgcctgaaaacatatgcccacctgttcgatgataaagtgatgaaacaactgaaacgccgtcgctataccggttggggtcgtctgagccgtaaactgattaatggtattcgtgataaacagagcggcaaaaccattctggattttctgaaaagtgatggctttgccaatcgcaattttatgcagctgattcatgatgatagcctgacctttaaagaggatattcagaaagcacaggttagcggtcagggtgatagtctgcatgaacatattgcaaatctggcaggtagtccggcaatcaaaaaaggtattctgcagaccgttaaagtggttgatgaactggttaaagttatgggtcgtcataaaccggaaaacatcgtgattgaaatggcacgtgaaaatcagaccacacagaaaggccagaaaaatagccgtgaacgcatgaaacgtattgaagaaggtattaaagaactgggcagccagattctgaaagaacatccggttgaaaatacccagctgcagaacgaaaaactgtatctgtattacctgcagaatggtcgtgacatgtatgttgatcaagaactggatattaatcgcctgagcgattatgatgtcgatcatattgttccgcagtcctttcttaaagatgatagcattgacaacaaagtgctgacccgtagcgacaaaaatcgtggtaaaagtgataatgtgccgagcgaagaggtggttaaaaagatgaaaaactattggcgtcaactgctgaacgccaaactgatcacacagcgcaaatttgataatctgacaaaagcagaacgtggtggtctgagcgagctggataaagcaggttttatcaaacgtcagctggttgaaacccgtcagatcacaaaacatgttgcacagattctggatagccgcatgaataccaaatatgacgaaaacgataaactgatccgcgaggttaaagtgattaccctgaaaagcaaactggtgagcgattttcgtaaagacttccagttttacaaagtgcgcgaaatcaacaattatcatcacgcacatgatgcctatctgaatgcagttgttggcaccgcactgattaagaaatatcctaaactggaaagcgagttcgtgtatggcgattataaagtttatgatgtgcggaagatgatcgccaaaagcgaacaagaaattggtaaagcaaccgccaaatacttcttctacagcaatattatgaacttcttcaaaaccgaaatcaccctggccaatggtgaaattcgtaaacgtccgctgatcgaaaccaatggcgaaaccggtgaaattgtttgggataaaggtcgtgattttgccaccgttcgtaaagttctgagtatgccgcaggttaacattgtgaaaaagaccgaagttcagaccggtggttttagcaaagaaagcattctgcctaaacgcaatagcgataagctgattgcccgtaaaaaagattgggacccgaaaaaatacggtggatttgatagcccgaccgttgcatatagcgttctggttgttgcaaaagtggaaaaaggtaagtccaagaaactgaaaagcgttaaagagctgctgggcattaccattatggaacgtagcagctttgaaaaaaacccgatcgatttcctggaagccaaaggttataaagaggtgaagaaagacctgattatcaagctgccaaagtatagcctgtttgaactggaaaatggtcgtaaacgcatgctggcaagtgcaggcgaactgcagaaaggtaatgaactggcactgccgagcaaatatgttaactttctgtatctggccagccactatgaaaagctgaaaggttcaccggaagataacgaacagaaacagttatttgtggaacagcacaaacactatctggatgaaattattgagcagatcagcgaatttagcaaacgtgttattctggcagatgccaatctggacaaagttctgtcagcatataacaaacaccgcgataaaccgattcgtgaacaggcagaaaacatcattcacctgtttaccctgaccaatctgggtgcaccggcagcctttaaatactttgataccaccattgatcgcaaacgttataccagcaccaaagaagttctggacgcaaccctgattcatcagagcattaccggtctgtatgaaacccgcattgatctgagccagttaggtggtgattgatgatgatgaaaaaaacgacaaagcagcactgattacagtgctgctttttttatccctgtgtggatgtggatgtggatgtggatggtaccttgacaattaatcatcggctcgtataatgtgtggaattgtgagcggataacaatttgttggaaccattcaaaacagcatagcaagttaaaataaggctagtccgttatcaacttgaaaaagtggcaccgagtcggtgcttttttgaaaaaaacgacaaagcagcactgattacagtgctgctttttttatccctgtgtggatgtggatgtggatgtggatcccgggttgacaattaatcatcggctcgtataatgtgtggaattgtgagcggataacaatttgttttagagctatgctgttttgaatggtcccaaaacaaaaaaaaaaaaaaaaaaaaaaaaaaagtcgttttagagctatgctgttttgaatggtcccaaaactgaaaaaaacgacaaagcagcactgattacagtgctgctttttttatccctgttctagaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcggccgcatatgagctctccggaagatctctcgaggtcgacgtacgtgcccatggatgcatttaattaattcgaagttaacttaagcttgtacactagtaaagttctgctatgtggcgcggtattatcccgtgttgacgccgggcaagagcaactcggtcgccgcatacactattctcagaatgacttggttgagtccatggcggccgccttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagctagatttcagtgcaatttatctcttcaaatgtagcacctgaagtcagccccatacgatataagttgtaattctcatgtttgacagcttatcatcgataagctccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatggcctgtcgcttgcggtattcggaatcttgcacgccctcgctcaagccttcgtcactggtcccgccaccaaacgtttcggcgagaagcaggccattatcgccggcatggcggccgacgcgctgggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattgtggaattgtgagcgg

SEQ ID NO: 44 - DNA, 7922 bp, synthetic construct
  source 1..7922: mol_type = other DNA
ctcgagataacgtggctttttttggtaagcagaaaataagtcattagtgaaaatatgtgtaggctggagctgcttcgaagttcctatactttctagagaataggaacttcggaataggaacttcaagatccccttattagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgagcttcaaaagcgctctgaagttcctatactttctagagaataggaacttcgaactgcaggtcgacggatccccggaattaattctcatgtttgacagagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattgtggaattgtgagcggttgacaattaatcatcggctcgtataatgtgtggaattgtgagcggataacaatttcacacaggaaacagaattcaggaggcaaaaatggacaaaaaatacagcattggtctggatattggcaccaatagcgttggttgggcagttattaccgatgaatataaagttccgagcaaaaaattcaaggtgctgggtaataccgatcgccatagcattaaaaagaatctgattggtgcactgctgttcgatagcggtgaaaccgcagaagcaacccgtctgaaacgtaccgcacgtcgtcgttatacccgtcgtaaaaatcgtatttgctatctgcaagagatctttagcaacgaaatggccaaagtggatgatagcttttttcatcgtctggaagaatcctttctggtggaagaagataaaaaacacgaacgccatccgatttttggcaacattgttgatgaagtggcctaccatgaaaaatatccgaccatttatcatctgcgcaaaaaactggttgatagcaccgataaagccgatctgcgtctgatttatctggcactggcacatatgatcaaatttcgtggtcattttctgatcgaaggtgatctgaatccggataatagtgatgtggataaactgttcattcagctggtgcagacctataatcagctgtttgaagaaaatccgattaatgccagcggtgttgatgcaaaagcaattctgagcgcacgtctgagcaaaagccgtcgcctggaaaacctgattgcacagctgcctggtgaaaaaaagaatggtctgtttggtaatctgatcgcactgagcctgggtctgaccccgaactttaaatcaaattttgatctggccgaagatgccaaactgcagctgagcaaagatacctatgatgatgatctggataatctgctggcacagattggtgatcagtatgcagacctgtttctggcagcgaaaaatctgagtgatgcaattctgctgagcgatattctgcgtgttaataccgaaattaccaaagcaccgctgagcgcaagtatgattaaacgttatgatgaacatcaccaggatctgaccctgctgaaagcactggttcgtcagcagctgccggaaaagtataaagaaatctttttcgaccagagcaagaatggctatgccggttatattgatggtggtgcaagccaagaagagttctacaaattcattaaaccgatcctggaaaagatggatggcaccgaagaactgctggttaaactgaatcgtgaagatctgctgcgtaaacagcgtacctttgataatggtagcattccgcatcagattcatctgggtgaactgcatgccattctgcgtcgtcaagaagatttttatccgttcctgaaagataaccgcgagaaaatcgaaaaaattctgacctttcgcatcccgtattatgtgggtccgctggcacgtggtaatagccgttttgcatggatgacccgtaaaagcgaagaaaccattacaccgtggaatttcgaagaggttgttgataaaggtgcaagcgcacagagctttattgaacgtatgaccaacttcgataaaaacctgccgaatgaaaaagttctgccgaaacatagcctgctgtatgaatatttcaccgtgtataatgagctgaccaaagtgaaatatgtgaccgaaggtatgcgtaaaccggcatttctgagcggtgaacagaaaaaagcgattgttgatctgctgtttaaaaccaatcgtaaggttaccgttaagcagctgaaagaggattatttcaaaaaaatcgagtgctttgatagcgtcgaaattagcggtgtggaagatcgttttaatgcaagcctgggcacctatcacgatctgctgaaaatcattaaagataaagattttctggacaacgaagagaacgaggacattctggaagatattgttctgacactgaccctgtttgaggatcgtgaaatgattgaagaacgcctgaaaacatatgcccacctgttcgatgataaagtgatgaaacaactgaaacgccgtcgctataccggttggggtcgtctgagccgtaaactgattaatggtattcgtgataaacagagcggcaaaaccattctggattttctgaaaagtgatggctttgccaatcgcaattttatgcagctgattcatgatgatagcctgacctttaaagaggatattcagaaagcacaggttagcggtcagggtgatagtctgcatgaacatattgcaaatctggcaggtagtccggcaatcaaaaaaggtattctgcagaccgttaaagtggttgatgaactggttaaagttatgggtcgtcataaaccggaaaacatcgtgattgaaatggcacgtgaaaatcagaccacacagaaaggccagaaaaatagccgtgaacgcatgaaacgtattgaagaaggtattaaagaactgggcagccagattctgaaagaacatccggttgaaaatacccagctgcagaacgaaaaactgtatctgtattacctgcagaatggtcgtgacatgtatgttgatcaagaactggatattaatcgcctgagcgattatgatgtcgatcatattgttccgcagtcctttcttaaagatgatagcattgacaacaaagtgctgacccgtagcgacaaaaatcgtggtaaaagtgataatgtgccgagcgaagaggtggttaaaaagatgaaaaactattggcgtcaactgctgaacgccaaactgatcacacagcgcaaatttgataatctgacaaaagcagaacgtggtggtctgagcgagctggataaagcaggttttatcaaacgtcagctggttgaaacccgtcagatcacaaaacatgttgcacagattctggatagccgcatgaataccaaatatgacgaaaacgataaactgatccgcgaggttaaagtgattaccctgaaaagcaaactggtgagcgattttcgtaaagacttccagttttacaaagtgcgcgaaatcaacaattatcatcacgcacatgatgcctatctgaatgcagttgttggcaccgcactgattaagaaatatcctaaactggaaagcgagttcgtgtatggcgattataaagtttatgatgtgcggaagatgatcgccaaaagcgaacaagaaattggtaaagcaaccgccaaatacttcttctacagcaatattatgaacttcttcaaaaccgaaatcaccctggccaatggtgaaattcgtaaacgtccgctgatcgaaaccaatggcgaaaccggtgaaattgtttgggataaaggtcgtgattttgccaccgttcgtaaagttctgagtatgccgcaggttaacattgtgaaaaagaccgaagttcagaccggtggttttagcaaagaaagcattctgcctaaacgcaatagcgataagctgattgcccgtaaaaaagattgggacccgaaaaaatacggtggatttgatagcccgaccgttgcatatagcgttctggttgttgcaaaagtggaaaaaggtaagtccaagaaactgaaaagcgttaaagagctgctgggcattaccattatggaacgtagcagctttgaaaaaaacccgatcgatttcctggaagccaaaggttataaagaggtgaagaaagacctgattatcaagctgccaaagtatagcctgtttgaactggaaaatggtcgtaaacgcatgctggcaagtgcaggcgaactgcagaaaggtaatgaactggcactgccgagcaaatatgttaactttctgtatctggccagccactatgaaaagctgaaaggttcaccggaagataacgaacagaaacagttatttgtggaacagcacaaacactatctggatgaaattattgagcagatcagcgaatttagcaaacgtgttattctggcagatgccaatctggacaaagttctgtcagcatataacaaacaccgcgataaaccgattcgtgaacaggcagaaaacatcattcacctgtttaccctgaccaatctgggtgcaccggcagcctttaaatactttgataccaccattgatcgcaaacgttataccagcaccaaagaagttctggacgcaaccctgattcatcagagcattaccggtctgtatgaaacccgcattgatctgagccagttaggtggtgattgatgatgatgaaaaaaacgacaaagcagcactgattacagtgctgctttttttatccctgtgtggatgtggatgtggatgtggatggtaccttgacaattaatcatcggctcgtataatgtgtggaattgtgagcggataacaatttgttggaaccattcaaaacagcatagcaagttaaaataaggctagtccgttatcaacttgaaaaagtggcaccgagtcggtgcttttttgaaaaaaacgacaaagcagcactgattacagtgctgctttttttatccctgtgtggatgtggatgtggatgtggatcccgggttgacaattaatcatcggctcgtataatgtgtggaattgtgagcggataacaatttgttttagagctatgctgttttgaatggtcccaaaacaaaaaaaaaaaaaaaaaaaaaaaaaaagtcgttttagagctatgctgttttgaatggtcccaaaactgaaaaaaacgacaaagcagcactgattacagtgctgctttttttatccctgttctagaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttacttattgtttgaaatcacgggggcgcaccgtataatttgaccgctttttgctcgag

SEQ ID NO: 45 - DNA, 18 bp, synthetic construct
  source 1..18: mol_type = other DNA
tccgcttcctttagcagc

SEQ ID NO: 46 - DNA, 18 bp, synthetic construct
  source 1..18: mol_type = other DNA
gctttacaggcttcgacg

SEQ ID NO: 47 - DNA, 18 bp, synthetic construct
  source 1..18: mol_type = other DNA
gctgggtaataccgatcg

SEQ ID NO: 48 - DNA, 20 bp, synthetic construct
  source 1..20: mol_type = other DNA
aattaccaaagcaccgctga

SEQ ID NO: 49 - DNA, 20 bp, synthetic construct
  source 1..20: mol_type = other DNA
cacctatcacgatctgctga

SEQ ID NO: 50 - DNA, 18 bp, synthetic construct
  source 1..18: mol_type = other DNA
gagcgaagaggtggttaa

SEQ ID NO: 51 - DNA, 20 bp, synthetic construct
  source 1..20: mol_type = other DNA
tgttgcaaaagtggaaaaag

SEQ ID NO: 52 - DNA, 20 bp, synthetic construct
  source 1..20: mol_type = other DNA
taatgtgtggaattgtgagc

SEQ ID NO: 53 - DNA, 18 bp, synthetic construct
  source 1..18: mol_type = other DNA
ggtgaacgctctcctgag

SEQ ID NO: 54 - DNA, 22 bp, synthetic construct
  source 1..22: mol_type = other DNA
ggttagactgttaacaacaacc

SEQ ID NO: 55 - DNA, 21 bp, synthetic construct
  source 1..21: mol_type = other DNA
catcacgtttttcactcctgc